(19) United States
(12) Patent Application Publication
(10) Pub. No.: US 2025/0182856 A1
(43) Pub. Date: Jun. 5, 2025

(54) CELL TYPE-SPECIFIC PREDICTION OF 3D CHROMATIN ARCHITECTURE

(71) Applicant: New York University, New York, NY (US)

(72) Inventors: Aristotelis Tsirigos, Hicksville, NY (US); Bo Xia, New York, NY (US); Jimin Tan, New York, NY (US)

(73) Assignee: New York University, New York, NY (US)

(21) Appl. No.: 18/843,202

(22) PCT Filed: Mar. 3, 2023
(86) PCT No.: PCT/US2023/014501

§ 371 Date: Aug. 30, 2024

Related U.S. Application Data
(60) Provisional application No. 63/316,873, filed on Mar. 4, 2022.

(51) Int. Cl. G16B 40/20; G06N 3/0455; G16B 15/10

(52) U.S. Cl. CPC G16B 40/20; G06N 3/0455; G16B 15/10

(57) ABSTRACT

The present disclosure relates to technologies for predicting genomic features, such as 3D genomic folding, in a target cell. Provided are methods for predicting genome structure and computer-implemented machines configured to predict genomic features. Wherein predicting 3D genomic features includes a neural network model architecture integrating (1) nucleotide-level DNA sequences, and (2) cell type-specific genomic features, wherein the cell type-specific genomic features comprise (i) genomic DNA-binding protein binding profile information, and (ii) chromatin accessibility information.

### CROSS-REFERENCE TO RELATED APPLICATION

This application claims the benefit under 35 U.S.C. § 119 (e) of U.S. Provisional Patent Application Ser. No. 63/316,873, filed Mar. 4, 2022, the entire contents of which is incorporated herein by reference.

### STATEMENT OF GOVERNMENT INTEREST

This invention was made with government support under Contract Nos. P01CA229086, R01CA252239, R01CA260028, and R01CA140729 awarded by the National Cancer Institute and National Institutes of Health to New York University School of Medicine and NYU Langone Health. The government has certain rights in the invention.

### TECHNICAL FIELD

The present disclosure relates generally to accurate cell type-specific prediction of 3D chromatin architecture.

### BACKGROUND

In mammalian cells, interphase chromosomes are hierarchically organized into large compartments which consist of multiple topologically associating domains (TADs) at the megabase and sub-megabase scale. Chromatin looping within TADs functions to restrict enhancer-promoter interactions at the kilobase scale of genes within the same TAD. The perturbation of TADs, such as through disruption of CTCF binding sites, can lead to aberrant chromatin interactions and changes in gene expression. As a result, mutations that disrupt 3D genome organization can substantially affect developmental programs and play important roles in genetic diseases and cancer.

The higher-order organization of the genome is largely determined by intrinsic DNA sequence features known as cis-elements that are bound by trans-acting factors in a sequence specific manner. For example, the location and orientation of CTCF binding sites act as a landmark for defining boundaries of TADs. Other factors, such as the cohesin proteins, act together to regulate chromatin via loop extrusion. While most TADs are conserved across cell types, a substantial amount (>10%) of TADs are dynamic and vary in different cells. In addition, widespread cell type-specific chromatin-looping contributes to the precise regulation of gene expression. These fine-scale chromatin interactions are controlled by chromatin remodeling proteins and cell type-specific transcription factors such as GATA1 and FOX1A. While the general organization of chromatin architecture is largely well described, the current challenge is to reveal the principles underlying cell type-specific chromatin folding. Chromatin architecture capture technologies, such as Hi-C, are used for examining chromatin-folding and functional studies of gene regulation at fine-scales and across cell types. However, these approaches can be costly and mostly require large cell numbers prohibiting their applications especially in the rare cell types.

Owing to its ability to detect complex interactions, deep learning has emerged as a powerful strategy for studying genomic features. Application of deep learning models could minimize the requirement for experimental analyses of chromatin architecture. Intrinsic features in DNA sequences of the genome partially determine its general folding principles, thus enabling prediction of genome organization. However, the differential compendium of trans-acting factors across cell types recognizes different combinations of DNA sequence features to establish cell type-specific chromatin interaction. Approaches that rely solely on DNA sequence for predictions lack the consideration of cell type-specific features and are unable to capture specific chromatin interactions in different cell types. Furthermore, methods that rely on chromatin data lack the consideration of DNA sequence features and thus undermine the capability for identifying critical DNA sequences that shape chromatin architecture.

An accurate prediction of cell type-specific chromatin folding requires a model which effectively recognizes and integrates both DNA sequence features and cell type-specific genomic information. A practical model should also minimize the requirement for input information without performance loss. Based on these principles, C.Origami was developed. C. Origami is a deep neural network that synergistically integrates DNA sequence features and two essential cell type-specific genomic features, DNA-binding protein profile (e.g., CTCF binding profile (CTCF ChIP-seq signal)) and chromatin accessibility information (e.g., ATAC-seq signal). C.Origami achieved accurate prediction of cell type-specific chromatin architecture in both normal and rearranged genomes. Additionally, the high-performance of C.Origami enables in silico genetic perturbation experiments that interrogate the impact on chromatin interactions and moreover, allows the identification of cell type-specific regulators of genomic folding through in silico genetic screening. Taken together, it is believed that the underlying deep learning architecture, Origami, to be generalizable for predicting genomic features and discovering novel genomic regulations

### SUMMARY

Presented herein is C.Origami, a neural network that accurately predicts cell type-specific genome folding, and enables in silico genetic studies of its regulation. C.Origami achieves cell type specificity by synergistically encoding both DNA sequence and minimum cell type-specific features. C.Origami is demonstrated to be able to de novo predict the genome folding of new cell types with high accuracy. Additionally, our model enables in silico genetic perturbation studies for discovering new cell type-specific regulators of genomic folding. Collectively, it is believed that the Origami architecture for integrating both DNA sequence information and cell type-specific features to be generalizable for future genomics studies, and is capable of discovering novel regulatory mechanisms. This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements. In one aspect, the present disclosure provides a method of predicting 3D genomic features in a target cell, the method comprising: training a neural network model architecture integrating (1) nucleotide-level DNA sequences, and (2) cell type-specific genomic features, wherein the cell type-specific genomic features comprise (i) genomic DNA-binding protein binding profile information, and (ii) chromatin accessibility information, thereby generating a trained neural network model architecture; applying the trained neural network model architecture to a genomic window of a target cell; and identifying genomic features within the genomic window of the target cell. In some embodiments, the nucleotide-level DNA sequences comprise a naturally occurring wild type sequence, a mutated DNA sequence, or a synthetic DNA sequence. In some embodiments, the cell type-specific genomic features comprise DNA binding profile information obtained for (1) transcription factor proteins, chromatin binding proteins, and chromatin-associated proteins, or from (2) chromatin feature distribution profiles. In some embodiments, the chromatin feature distribution profiles comprise histone modifications, DNA modifications, chromatin accessibility information. In some embodiments, the genomic DNA-binding protein is selected from the group consisting of CTCF, CTCFL, RAD21, STAG1, STAG2, SMC1, SMC3, ZNF143, YY1, NIPBL, WAPL, TRIM22, and BATF. In some embodiments, the genomic DNA-binding protein is CTCF. In some embodiments, the genomic DNA-binding protein binding profile information comprises ChIP-seq data, CUT&RUN data, CUT&TAG data, or DamID data in the genomic window of the target cell. In some embodiments, the cell type-specific genomic features comprise chromatin feature distribution profiles. In some embodiments, the chromatin feature distribution profiles comprise histone modification data, DNA modification data. In some embodiments, the chromatin accessibility information comprises one or more of H3K4ac, H3K9ac, H3K27ac, H3K4mel, H3K4me2, H3K4me3, H3K9me3, H3K27me3, H3K36me3. In some embodiments, the chromatin accessibility information is selected from the group consisting of ATAC-seq data, DNase-seq data, or MNase-seq data. In some embodiments, the cell type-specific genomic comprises a DNA modification profile. In some embodiments, the DNA modification profile comprises DNA methylated cytosine (5mC), DNA hydroxylmethylaed cytosine (5hmC), or DNA formylated cytosine (5hmC), or carboxylated cytosine (5caC). In some embodiments, the chromatin accessibility information comprises ATAC-seq data in the genomic window of the target cell. In some embodiments, genomic features comprise identification of a topologically associating domain (TAD). In some embodiments, the genomic window comprises a contiguous genomic region of 2 million bases. In some embodiments, the model architecture comprises two encoders, a transformer module, and a decoder. In some embodiments, the decoder is a decoder associated with Hi-C contact matrices for predicting complex chromatin architecture.

In one aspect, the present disclosure provides a computer-implemented machine for predicting 3D genomic features in a target cell, comprising: a processor; a neural network comprising a first encoder, a second encoder, a transformer module, and a decoder; and a tangible computer-readable medium operatively connected to the processor and including computer code configured to: train a neural network model architecture integrating (1) nucleotide-level DNA sequences, and (2) cell type-specific genomic features, wherein the cell type-specific genomic features comprise (i) genomic DNA-binding protein binding profile information, and (ii) chromatin accessibility information, thereby generating a trained neural network model architecture; apply the trained neural network model architecture to a genomic window of a target cell; and identify genomic features within the genomic window of the target cell. In some embodiments, the nucleotide-level DNA sequences comprise a naturally occurring wild type sequence, a mutated DNA sequence, or a synthetic DNA sequence. In some embodiments, the cell type-specific genomic features comprise DNA binding profile information obtained for (1) transcription factor proteins, chromatin binding proteins, and chromatin-associated proteins, or from (2) chromatin feature distribution profiles. In some embodiments, the chromatin feature distribution profiles comprise histone modifications, DNA modifications, chromatin accessibility information. In some embodiments, the genomic DNA-binding protein is selected from the group consisting of CTCF, CTCFL, RAD21, STAG1, STAG2, SMC1, SMC3, ZNF143, YY1, NIPBL, WAPL, TRIM22, and BATF. In some embodiments, the genomic DNA-binding protein is CTCF. In some embodiments, the genomic DNA-binding protein binding profile information comprises ChIP-seq data, CUT&RUN data, CUT&TAG data, or DamID data in the genomic window of the target cell. In some embodiments, the cell type-specific genomic features comprise chromatin feature distribution profiles. In some embodiments, the chromatin feature distribution profiles comprise histone modification data, DNA modification data. In some embodiments, the chromatin accessibility information comprises one or more of H3K4ac, H3K9ac, H3K27ac, H3K4mel, H3K4me2, H3K4me3, H3K9me3, H3K27me3, H3K36me3. In some embodiments, the chromatin accessibility information is selected from the group consisting of ATAC-seq data, DNase-seq data, or MNase-seq data. In some embodiments, the cell type-specific genomic comprises a DNA modification profile. In some embodiments, the DNA modification profile comprises DNA methylated cytosine (5mC), DNA hydroxylmethylaed cytosine (5hmC), or DNA formylated cytosine (5hmC), or carboxylated cytosine (5caC). In some embodiments, the genomic DNA-binding protein binding profile information comprises chIP-seq data for the genomic DNA-binding protein in the genomic window of the target cell. In some embodiments, the chromatin accessibility information comprises ATAC-seq data in the genomic window of the target cell. In some embodiments, genomic features comprise identification of a topologically associating domain (TAD). In some embodiments, the genomic window comprises a contiguous genomic region of 2 million bases. In some embodiments, the model architecture comprises two encoders, a transformer module, and a decoder. In some embodiments, the decoder is a decoder associated with Hi-C contact matrices for predicting complex chromatin architecture.

In some embodiments, genomic features comprise genome organization. In some embodiments, genomic features comprise genome folding.

### BRIEF DESCRIPTION OF THE FIGURES

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

FIGS. 1A and 1B show de novo prediction of cell type-specific genomic features with Origami. FIG. 1A is a schematic of generalized Origami architecture. Origami adopts an encoder-decoder design, separately encoding DNA sequence features and cell type-specific genomic features. The two streams of encoded information are concatenated and processed by a transformer module. The decoder converts the processed 1D information to the final prediction, such as a Hi-C interaction matrix. FIG. 1B shows applying Origami model to predicting the Hi-C interaction matrix. The best-practice model integrates DNA sequence, CTCF ChIP-seq signal and ATAC-seq signal as input features to predict Hi-C interaction matrix in 2 Mb windows.

FIGS. 2A-2H illustrate how C.Origami accurately predicts chromatin structure. FIGS. 2A-2B show experimental Hi-C matrices (FIG. 2A) and C.Origami predicted Hi-C matrices (FIG. 2B) of IMR-90 cell line at chromosome 2 (left), chromosome 10 (middle), and chromosome 15 (right), representing training, validation and test chromosomes, respectively. FIG. 2C shows input CTCF binding profiles and chromatin accessibility profiles. FIG. 2D shows insulation scores calculated from experimental Hi-C matrices (solid line) and C.Origami predicted Hi-C matrices (dotted line). Pearson correlation coefficients comparing the insulation were indicated in the plots. FIG. 2E shows the insulation correlation between predicted and experimental Hi-C matrices across all windows in both validation and test chromosomes. Each group included both Pearson correlation (r) and Spearman correlation (p) coefficients. FIG. 2F shows the distribution of experimental Hi-C intensity scores by insulation correlation (Pearson's r) between prediction and experiment. Each point represents a 2 Mb genomic window in chromosome 15 (test). Colormap indicates the Spearman's p of insulation correlation between prediction and experiment. FIG. 2G shows the average intensity of the interaction matrix across genomic distances. FIG. 2H shows the distance-stratified interaction correlation (Pearson) between prediction and experiment

FIGS. 3A-3G illustrate cell type-specific de novo prediction of chromatin structure. FIG. 3A shows experimental Hi-C matrices from IMR-90 (left) and GM12878 (middle) cell lines at chromosome 2, highlighting cell type-specific chromatin differences (right). FIG. 3B shows C.Origami-predicted Hi-C matrices of IMR-90 (left) and GM12878 (middle), precisely recapitulated the experimental Hi-C matrices (FIG. 3A). The arrow heads highlighted differential chromatin interactions between the two cell types. FIG. 3C shows CTCF binding profiles and chromatin accessibility profiles of IMR-90 (left), GM12878 (middle) and their difference (right). FIG. 3D shows insulation scores calculated from experimental Hi-C matrices (solid line) and C.Origami predicted Hi-C matrices (dotted line) of IMR-90 (left), GM12878 (middle) and their difference (right). FIG. 3E shows the distribution of interaction intensity by insulation correlation (Pearson) between the experimental Hi-C matrices of IMR-90 and GM12878. Colormap indicates the corresponding Spearman correlation coefficient (p). Dotted lines denote the filtering criteria in selecting representative loci with cell-type specificity. FIG. 3F shows the Pearson correlation between insulation scores calculated from predicted and experimental Hi-C matrices across cell types. Prediction from each cell type was similar to the corresponding experimental data. FIG. 3G shows Pearson's r of predicted insulation difference and experimental insulation difference between IMR-90 and other cell types. The correlation was calculated as: Pearson (Insu(IMR-90_pred)-Insu(Target_pred), Insu(IMR-90_data)-Insu(Target_data)). High correlation indicates that our model detected cell types-specific features applicable across different cell types.

FIGS. 4A-4F illustrate how C.Origami enables allele-specific prediction of 3D chromatin architecture in rearranged cancer genome. FIG. 4A shows chromosomal translocation between chromosome 7 and chromosome 9 in CUTLL1 T cell leukemia cells. FIG. 4B shows experimental Hi-C data mapped to a custom reference chromosome with t(7,9) translocation. FIGS. 4C-4D show C.Origami prediction of chromatin architecture of chromosome 7 (FIG. 4C) and chromosome 9 (FIG. 4D) in CUTLLI cells. The windows represented intact chromosomal loci around the translocation sites in CUTLLI cells. FIG. 4E shows C.Origami prediction of chromatin architecture at the t(7,9) translocation locus. FIG. 4F shows a simulated Hi-C contact matrix using prediction for mimicking of experimental mapping results. The simulated result was averaged from the prediction of both normal and translocated alleles. The simulated Hi-C matrix was aligned to the experimental Hi-C matrix (FIG. 4B), and highlighted the neo-TAD at the translocation locus. Black arrow head indicates the translocation site. The grey arrow head indicates a stripe in the neo-TAD.

FIGS. 5A-5F illustrate in silico genetic experiments for identifying cis-regulatory elements determining chromatin architecture. FIG. 5A is a schematic of in silico deletion and masked mutation experiments. A deletion experiment completely removed both DNA sequences and genomic signals, while a masked mutation experiment shuffled DNA sequence but not the genomic peaks and their underlying DNA sequences. FIG. 5B shows a 500bp deletion in chromosome 8 led to chromatin looping changes in T cells. The presented 2 Mb window starts at the promoter region of MYC, and the experimental deletion perturbed a CTCF binding site at the arrowhead location. The presented results include C.Origami prediction of the Hi-C contact matrices with (middle) or without (left) the deletion, and their difference (right). The virtual 4C signal, calculated from the predicted Hi-C matrices, is shown at the bottom. FIG. 5C is a schematic of impact score that indicates how perturbation of one locus affected the local chromatin folding, and sensitivity score that indicates how sensitive a locus is to genetic perturbations in neighboring areas. FIG. 5D shows GRAM score, indicating the contribution of genomic location to the predicted Hi-C matrix. FIGS. 5E-5F show sliding-window deletion screening (FIG. 5E) and CTCF-masked mutation screening (FIG. 5F) across a 2 Mb window corresponding to FIG. 5D. Impact and sensitivity scores were shown on the horizontal and vertical axis, respectively. CTCF peak and its DNA sequences were masked to prevent disruption of CTCF signal.

FIGS. 6A-6D illustrate how genome-wide in silico screening uncovers trans-regulators of chromatin folding. FIG. 6A is a schematic of whole-genome in silico screening process. FIG. 6B is a heatmap of weighted scores across the four categories of in silico screen-determined contributing factors. The plot highlights three major clusters of contributing factors. FIGS. 6C-6D show in silico identified contributing factors ranked by their weighted scores in each of the four categories as defined in FIG. 6B.

FIG. 7 shows C.Origami model structure and module components. A detailed schematic of C.Origami model architecture. The DNA encoder and Genomic Feature encoder have similar architectures and they only different in input channels where DNA encoder has 5 and Feature encoder has 2. To encoder data, the encoder was built with 12 convolution blocks, each consisting of a scaling module and residual module. The scaling module downscales input features by a factor of 2 with a stride-2 1D convolution layer. The residual module promotes information propagation in very deep networks (REF Deep Residual Learning for Image Recognition). The number of modules was carefully chosen such that the 2,097,152 input is scaled down to 256 bins at the end of the encoder. To enhance interactions within the 2 mb window, an attention module was used that consists of 8 attention blocks modified from the transformer architecture. Each position of the output is concatenated with every other position to form a 2D matrix, resembling a vector outer-product process. To refine the final prediction, a 5-layer dilated 2D convolutional network was used as decoder. The dilation parameters were selected to ensure that every position at the last layer has a receptive field covering the input range.

FIGS. 8A-8B illustrate the performance of C.Origami trained with DNA sequence and CTCF profiles. While C.Origami with DNA sequence and CTCF profile as inputs achieved good performance in validation and test set in IMR-90 (FIG. 8A), but it performed poorly in de novo GM12878 prediction (FIG. 8B).

FIGS. 9A-9F illustrate C.Origami trained with DNA sequence, CTCF profile, and chromatin accessibility profiles performed better. FIGS. 9A-9C show experiment at chr2:400,000-2,497,152, comparing IMR-90 and GM12878 ground truth (FIG. 9A) among predictions of sequence +CTCF (FIG. 9B) and sequence+CTCF+ATAC-seq (FIG. 9C). FIGS. 9D-9F show a similar experiment at chr10:122,700,000-122,797,152.

FIGS. 10A-10C illustrate an ablation study on different input features. In FIG. 10A, DNA sequences are randomly shuffled at base pair level. From left to right, reference prediction with all inputs (left), prediction with sequence shuffled (middle), difference between perturbed prediction and reference prediction (right). In FIG. 10B, CTCF signal is randomly shuffled. In FIG. 10C, ATAC-seq signal is randomly shuffled.

FIG. 11 shows chromosome karyotype with chromosome wide intensity and insulation score correlation. chromosome 1 to chromosome X are plotted to visualize the insulation score correlation between prediction and experimental Hi-C. Average intensity of 2 Mb windows are plotted in red. Telomere and centromere regions are denoted with red segments on the genome.

FIGS. 12A-12C show fusing C.Origami-predicted 2 Mb Hi-C maps into larger interaction maps. Shown are fused maps spanning 5 Mb (FIG. 12A), 10 Mb (FIG. 12B), and 50 Mb (FIG. 12C) on chromosome 15 starting at 40 Mb.

FIGS. 13A-13B show C.Origami predicts chromatin folding features across multiple cell types. Prediction and experimental Hi-C in two loci-Chr12:89,300,000-91,397,152 (FIG. 13A) and Chr20:47,000,000-49,097,152 (FIG. 13B)-are presented across IMR-90, GM12878, H1-hESCs, and K562.

FIGS. 14A-14G show genome-wide statistics on cell type-specific prediction performance. FIG. 14A shows Pearson's r and FIG. 14B shows Spearman's p between prediction (row) and ground truth (column) for different cell types with insulation score and observed/expected score as metrics. The scores are calculated based on the differentially structured loci defined in FIG. 3. The correlation between Observed/Expected contact matrices was lower due to higher background noise. FIG. 14C shows Pearson's r and FIG. 14D shows Spearman's p value of prediction difference and ground truth difference for different cell types: Correlation(Insu(Celltype1_pred)-Insu(Cell_type2_pred), Insu(Cell_type1_data)-Insu(Cell_type2_data)) FIG. 14E shows selecting structurally conserved loci across different cell types. Conserved subset accounting for ˜60% of the data. FIG. 14F shows Pearson's r and FIG. 14G shows Spearman's p between insulation scores of prediction and ground truth in the conserved subset.

FIGS. 15A-15B show images comparing cell-type specific prediction performance of C.Origami with Akita. Two loci are presented—Chr5 (FIG. 15A) and Chr2 (FIG. 15B). Each locus includes the prediction in IMR-90 cells and GM12878 cells. C.Origami outperforms Akita in cell type-specific chromatin folding prediction.

FIGS. 16A-16B show a performance comparison of C.Origami models trained with sparse information and dense information. Two loci are presented—Chr3:158,600,000-160,697,152 (FIG. 16A) and Chr11:85,100,000-87,197,152 (FIG. 16B). Each locus includes data and predictions on IMR-90 cells and GM12878 cells, and their difference.

FIGS. 17A-17D show mouse prediction. Two loci are presented—Chr2 and Chr16. Each locus includes data and predictions on IMR-90 cells and GM12878 cells, as well as the difference between the two.

FIGS. 18A-18B show in silico genetic experiments performed on IMR-90 cells. Two in silico deletion experiment were represented presented-660,000-676,384 (FIG. 18A) and 127,720,000-127,736,384 (FIG. 18B). Each experiment includes the prediction before (left) and after deletion (middle). The difference in chromatin folding after deletion were presented on the right.

FIG. 19 illustrates a computer system for use with certain implementations.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

The mammalian genome is spatially organized in the nucleus to enable cell type-specific gene expression. Investigating how chromatin architecture determines this specificity remains a big challenge. To measure the 3D chromatin architecture of a specific cell type, complex chromosomal conformation capturing technique such as Hi-C is required. However, the high cost and technical limitations of the method restricts its application, particularly in the context of cancer genomes. Accordingly, there is a pressing need for technologies that enable in silico prediction of genomic architecture.

### I. Methods for Predicting 3D Chromatin Structure

Provided herein are methods for predicting chromatin structure in a target cell. In some embodiments, the provided methods enable the prediction of 3D chromatin architecture within a target cell.

Provided herein are methods of predicting 3D genomic features in a target cell. In some embodiments, genomic features comprise genome organization, including 3D genome organization. In some embodiments, genomic features comprise genome folding. In some embodiments, the method comprises training a neural network model architecture integrating genomic structure data, epigenomic data, and/or genomic sequence data. Genomic structure data can include, for example, chromatin folding data, topological associating domain (TADs) and TAD boundary data, and other known metrics for assessing genome structure, including 3D chromatin structure. Genomic sequence data generally includes genomic DNA sequence data, such as continuous sequences of DNA within a chromosome. The genomic sequence data can be obtained by applying known genomic DNA sequencing methods to a target cell, or from previously-generated genomic DA sequence data, such as from a genomic DNA sequence database. Epigenomic data can include, for example, transcriptional regulatory data, such as genomic DNA-binding protein data (e.g., CTCF-binding data). In some embodiments, the epigenomic data is obtained for a genomic window of a target cell

In some embodiments, a neural network model architecture integrates nucleotide-level DNA sequences, and/or cell type-specific genomic features. In some embodiments, a neural network model architecture integrates nucleotide-level DNA sequences and cell type-specific genomic features. DNA sequences can include a wild type sequence, a mutated DNA sequence, or a synthetic DNA sequence. Cell type-specific features can include one or more of genomic DNA-binding protein binding profile information, and chromatin accessibility information. Cell type-specific features can include DNA binding profile information obtained for transcription factor proteins, chromatin binding proteins, and chromatin-associated proteins, or from chromatin feature distribution profiles. Chromatin feature distribution profiles can include data describing histone modifications, DNA modifications, chromatin accessibility information.

Genomic DNA-binding protein binding profile information can include ChIP-sequencing (ChIP-seq) data, CUT&RUN data, CUT&TAG data, or DamID data obtained for a genomic DNA-binding protein in a target cell. Non-limiting examples of genomic DNA-binding proteins include CCCTC-binding factor (CTCF), CTCFL, RAD21, STAGI, STAG2, SMC1, SMC3, ZNF143, YY1, NIPBL, WAPL, TRIM22, and BATF. In some preferred embodiments, the genomic DNA-binding protein is CTCF. In some embodiments, the genomic DNA-binding protein binding profile information can include CTCF ChIP-seq data obtained for a genomic DNA-binding protein in a target cell. In some embodiments, the genomic DNA-binding protein binding profile information can include RAD21 ChIP-seq data obtained for a genomic DNA-binding protein in a target cell. In some embodiments, the genomic DNA-binding protein binding profile information can include STAG1 ChIP-seq data obtained for a genomic DNA-binding protein in a target cell. In some embodiments, the genomic DNA-binding protein binding profile information can include SMC3 ChIP-seq data obtained for a genomic DNA-binding protein in a target cell. In some embodiments, the genomic DNA-binding protein binding profile information can include CTCFL ChIP-seq data obtained for a genomic DNA-binding protein in a target cell. In some embodiments, the genomic DNA-binding protein binding profile information can include STAG2 ChIP-seq data obtained for a genomic DNA-binding protein in a target cell. In some embodiments, the genomic DNA-binding protein binding profile information can include SMC1 ChIP-seq data obtained for a genomic DNA-binding protein in a target cell. In some embodiments, the genomic DNA-binding protein binding profile information can include ZNF143 ChIP-seq data obtained for a genomic DNA-binding protein in a target cell. In some embodiments, the genomic DNA-binding protein binding profile information can include YY1 ChIP-seq data obtained for a genomic DNA-binding protein in a target cell. In some embodiments, the genomic DNA-binding protein binding profile information can include NIPBL ChIP-seq data obtained for a genomic DNA-binding protein in a target cell. In some embodiments, the genomic DNA-binding protein binding profile information can include WAPL ChIP-seq data obtained for a genomic DNA-binding protein in a target cell. In some embodiments, the genomic DNA-binding protein binding profile information can include TRIM22 ChIP-seq data obtained for a genomic DNA-binding protein in a target cell. In some embodiments, the genomic DNA-binding protein binding profile information can include BATF ChIP-seq data obtained for a genomic DNA-binding protein in a target cell.

Chromatin accessibility information can include Assay for Transposase-Accessible Chromatin (ATAC) sequencing (ATAC-seq) data, DNase-seq data, or MNase-seq data. DNase-seq data, or MNase-seq data. In some prefereed embodiments, chromatin accessibility information can include Assay for Transposase-Accessible Chromatin (ATAC) sequencing (ATAC-seq) data In some embodiments, chromatin accessibility data can include one or more of acetylated H3K4 (H3K4ac), acetylated H3K9 (H3k9ac), acetylated H3K27 (H3K27ac), H3K4mel, H3K4me2, H3K4me3, H3K9me3, H3K27me3, H3K36me3, and data describing the same. In some embodiments, the chromatin accessibility data is Chip-seq data.

In some embodiments, cell type-specific genomic comprises a DNA modification profile. In some embodiments, the DNA modification profile comprises DNA methylated cytosine (5mC), DNA hydroxylmethylaed cytosine (5hmC), or DNA formylated cytosine (5hmC), or carboxylated cytosine (5caC).

In some embodiments, a trained neural network is applied to a genomic window in a target cell. As used herein, the term “genomic window” refers to a contiguous segment of genomic DNA. A genomic window can contain at least 100 bases, at least 1000 bases, at least 10000 bases, at least 100000 bases, at least 1 million bases, at least 1.5 million bases, at least 2 million bases, at least 2.5 million bases, at least 3 million bases, at least 4 million bases, or at least 5 million bases. In some embodiments, a genomic window comprises 1 million bases, 1.1 million bases, 1.2 million bases, 1.3 million bases, 1.4 million bases, 1.5 million bases, 1.6 million bases, 1.7 million bases, 1.8 million bases, 1.9 million bases, 2 million bases, 2.1 million bases, 2.2 million bases, 2.3 million bases, 2.4 million bases, or 2.5 million bases. In some embodiments, a genomic window comprises about 2 million bases. In some embodiments, a genomic window comprises 2 million bases.

Provided is a method of predicting 3D genomic features in a target cell, the method comprising training a neural network model architecture integrating (1) nucleotide-level DNA sequences, and (2) cell type-specific genomic features, wherein the cell type-specific genomic features comprise (i) genomic DNA-binding protein binding profile information, and (ii) chromatin accessibility information, thereby generating a trained neural network model architecture; applying the trained neural network model architecture to a genomic window of a target cell; and identifying genomic features within the genomic window of the target cell. In some embodiments, predicting genomic features comprise identifying or characterizing a topologically associated domain (TAD).

### II. Computer Implemented Machines for Predicting 3D Chromatin Structure

Provided herein are computer implemented machines for predicting chromatin structure in a target cell. In some embodiments, the provided machines enable the prediction of 3D chromatin architecture within a target cell.

Provided herein are computer-implemented machines for predicting 3D genomic features in a target cell. In some embodiments, a machine comprises at least one processor, a neural network, and a tangible computer-readable medium operatively connected to the processor. In some embodiments, the neural network comprises a first encoder, a second encoder, a transformer module, and a decoder. In some embodiments, the tangible computer-readable medium includes computer code. In some embodiments, the model architecture comprises two encoders, a transformer module, and a decoder. In some embodiments, the decoder is a decoder associated with Hi-C contact matrices for predicting complex chromatin architecture.

In some embodiments, a computer implemented machine described herein is configured to: train a neural network model architecture integrating (1) nucleotide-level DNA sequences, and (2) cell type-specific genomic features, wherein the cell type-specific genomic features comprise (i) genomic DNA-binding protein binding profile information, and (ii) chromatin accessibility information, thereby generating a trained neural network model architecture; apply the trained neural network model architecture to a genomic window of a target cell; and identify genomic features within the genomic window of the target cell.

In some embodiments, a computer implemented machine is configured to train a neural network model architecture integrating nucleotide-level DNA sequences, and/or cell type-specific genomic features. In some embodiments, a neural network model architecture integrates nucleotide-level DNA sequences and cell type-specific genomic features. Cell type-specific features can include one or more of genomic DNA-binding protein binding profile information, and chromatin accessibility information.

Genomic DNA-binding protein binding profile information can include ChIP-sequencing (ChIP-seq) data obtained for a genomic DNA-binding protein in a target cell. Non-limiting examples of genomic DNA-binding proteins include CCCTC-binding factor (CTCF), CTCFL, RAD21, STAGI, STAG2, SMC1, SMC3, ZNF143, YY1, NIPBL, WAPL, TRIM22, and BATF. In some preferred embodiments, the genomic DNA-binding protein is CTCF. In some embodiments, the genomic DNA-binding protein binding profile information can include CTCF ChIP-seq data obtained for a genomic DNA-binding protein in a target cell. In some embodiments, the genomic DNA-binding protein binding profile information can include RAD21 ChIP-seq data obtained for a genomic DNA-binding protein in a target cell. In some embodiments, the genomic DNA-binding protein binding profile information can include STAG1 ChIP-seq data obtained for a genomic DNA-binding protein in a target cell. In some embodiments, the genomic DNA-binding protein binding profile information can include SMC3 ChIP-seq data obtained for a genomic DNA-binding protein in a target cell. In some embodiments, the genomic DNA-binding protein binding profile information can include CTCFL ChIP-seq data obtained for a genomic DNA-binding protein in a target cell. In some embodiments, the genomic DNA-binding protein binding profile information can include STAG2 ChIP-seq data obtained for a genomic DNA-binding protein in a target cell. In some embodiments, the genomic DNA-binding protein binding profile information can include SMC1 ChIP-seq data obtained for a genomic DNA-binding protein in a target cell. In some embodiments, the genomic DNA-binding protein binding profile information can include ZNF143 ChIP-seq data obtained for a genomic DNA-binding protein in a target cell. In some embodiments, the genomic DNA-binding protein binding profile information can include YY1 ChIP-seq data obtained for a genomic DNA-binding protein in a target cell. In some embodiments, the genomic DNA-binding protein binding profile information can include NIPBL ChIP-seq data obtained for a genomic DNA-binding protein in a target cell. In some embodiments, the genomic DNA-binding protein binding profile information can include WAPL ChIP-seq data obtained for a genomic DNA-binding protein in a target cell. In some embodiments, the genomic DNA-binding protein binding profile information can include TRIM22 ChIP-seq data obtained for a genomic DNA-binding protein in a target cell. In some embodiments, the genomic DNA-binding protein binding profile information can include BATF ChIP-seq data obtained for a genomic DNA-binding protein in a target cell.

Chromatin accessibility information can include Assay for Transposase-Accessible Chromatin (ATAC) sequencing (ATAC-seq) data, DNase-seq data, or MNase-seq data. DNase-seq data, or MNase-seq data. In some prefereed embodiments, chromatin accessibility information can include Assay for Transposase-Accessible Chromatin (ATAC) sequencing (ATAC-seq) data In some embodiments, chromatin accessibility data can include one or more of acetylated H3K4 (H3K4ac), acetylated H3K9 (H3k9ac), acetylated H3K27 (H3K27ac), H3K4mel, H3K4me2, H3K4me3, H3K9me3, H3K27me3, H3K36me3, and data describing the same. In some embodiments, the chromatin accessibility data is Chip-seq data.

In some embodiments, cell type-specific genomic comprises a DNA modification profile. In some embodiments, the DNA modification profile comprises DNA methylated cytosine (5mC), DNA hydroxylmethylaed cytosine (5hmC), or DNA formylated cytosine (5hmC), or carboxylated cytosine (5caC).

In some embodiments of a computer implemented machine as described herein, a trained neural network is applied to a genomic window in a target cell. As used herein, the term “genomic window” refers to a contiguous segment of genomic DNA. A genomic window can contain at least 100 bases, at least 1000 bases, at least 10000 bases, at least 100000 bases, at least 1 million bases, at least 1.5 million bases, at least 2 million bases, at least 2.5 million bases, at least 3 million bases, at least 4 million bases, or at least 5 million bases. In some embodiments, a genomic window comprises 1 million bases, 1.1 million bases, 1.2 million bases, 1.3 million bases, 1.4 million bases, 1.5 million bases, 1.6 million bases, 1.7 million bases, 1.8 million bases, 1.9million bases, 2 million bases, 2.1 million bases, 2.2 million bases, 2.3 million bases, 2.4 million bases, or 2.5 million bases. In some embodiments, a genomic window comprises about 2 million bases. In some embodiments, a genomic window comprises 2 million bases.

### III. C.Origami

Described herein is one embodiment of a deep neural network model that accurately predicts cell type-specific chromatin by incorporating DNA sequence, CTCF binding, and chromatin accessibility profiles, referred to as C.Origami. Origami enables in silico experiments that examine the impact of genetic perturbations on chromatin interactions, and moreover, leads to the identification of a compendium of cell type-specific regulators of 3D chromatin architecture. It is further belived that Origami-the underlying model architecture of C.Origami-to be generalizable for future genomics studies in discovering novel gene regulatory mechanisms.

### Origami: A Model for Predicting Cell Type-Specific Genomic Features

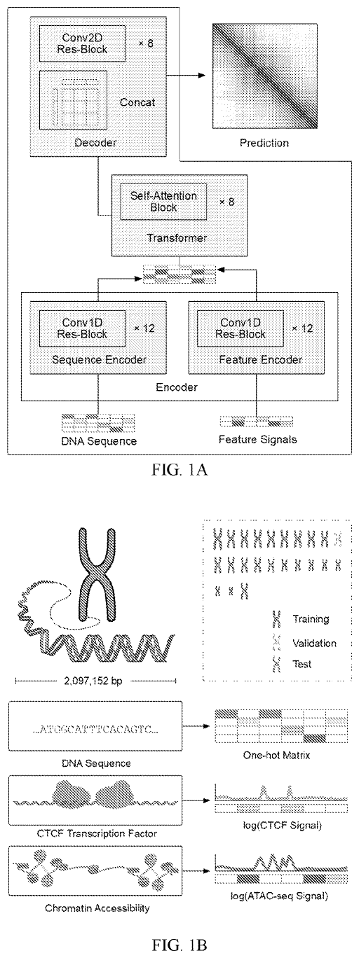

To achieve accurate and cell type-specific prediction of genomic features, we first developed a generalizable model architecture we named Origami to synergistically integrate both nucleotide-level DNA sequence and cell type-specific genomic signal (FIG. 1A). The former enables recognition of informative sequence motifs, while the later provides cell type-specific features. The Origami architecture consists of two encoders, a transformer module and a decoder (FIG. 1A, see below). The two encoders process DNA sequence and genomic features independently. The encoded features are concatenated and further processed by a transformer, which allows the encoded information to exchange freely between different genomic regions. The decoder in Origami synthesizes the processed information to make predictions, and depending on the task, can be customized to specific downstream prediction types. In this study, we deployed a decoder for predicting complex chromatin architecture, Hi-C contact matrices, and therefore named this variant C.Origami.

To cover typical TADs in the genome while maximizing computation efficiency, C.Origami focused on 2 mega-base (2 Mb) sized genomic windows. Each encoder consists of twelve 1D convolution blocks to reduce features from 2 Mb locus down to 256 bins with a bin size of 8,192bp (see Methods). DNA sequence and genomic features within the 2 Mb window were separately encoded as nucleotide-level features (FIG. 1B, see Methods). Hi-C matrix from the corresponding 2 Mb genomic window was processed to have the same bin size of 8,192 bp. To train the model, we used data from IMR-90, a fibroblast cell line isolated from normal lung tissue, and randomly split the chromosomes into training, validation (chromosome 10), and test (chromosome 15) sets.

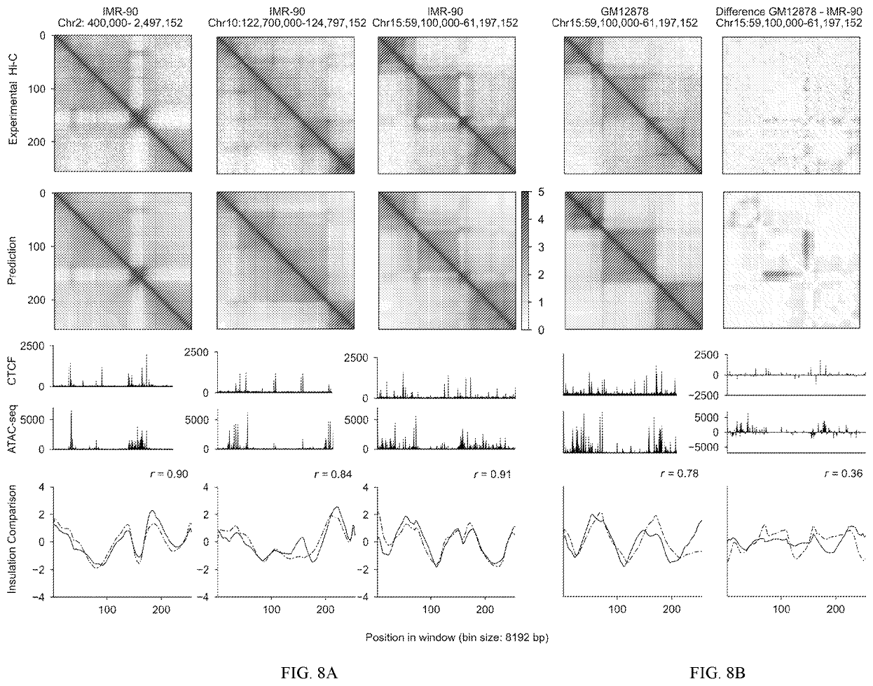

To select genomic features as input for cell type-specific of chromatin architecture, we considered three criteria: 1) representativeness for cell type-specific identity; 2) general accessibility and experimental robustness; 3) a minimum number of features to enable broad applicability of the model. CTCF binding is one of the most critical determinants of 3D chromatin architecture, thus we initially trained the model using DNA sequences and CTCF ChIP-seq signals as the only cell type-specific genomic feature (FIGS. 8A-8B). Our model performed well in most predictions, capturing the TAD structures and chromatin interaction events (FIGS. 8A-8B). However, we found the prediction did not recognize some fine-scale chromatin interaction features, especially in de novo prediction on a cell type (FIGS. 8A-8B). These results indicate that integrating DNA sequence with CTCF binding signal alone is not sufficient for optimum prediction of cell type-specific 3D genome conformation.

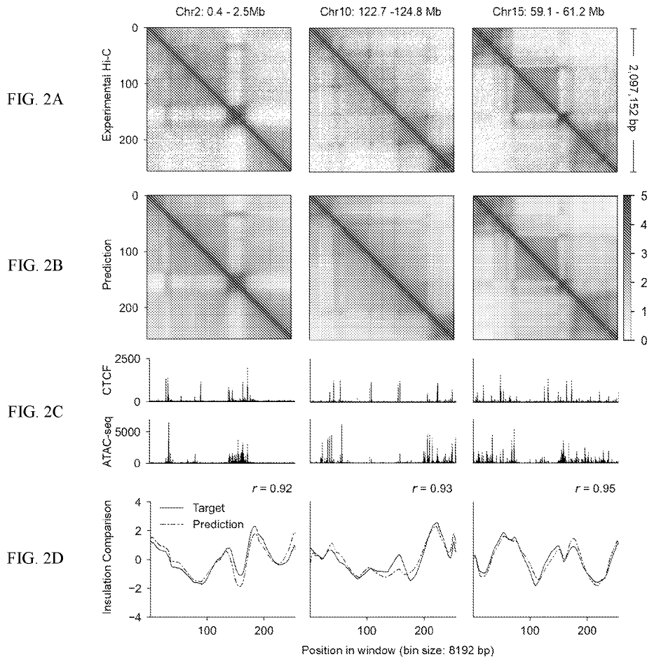

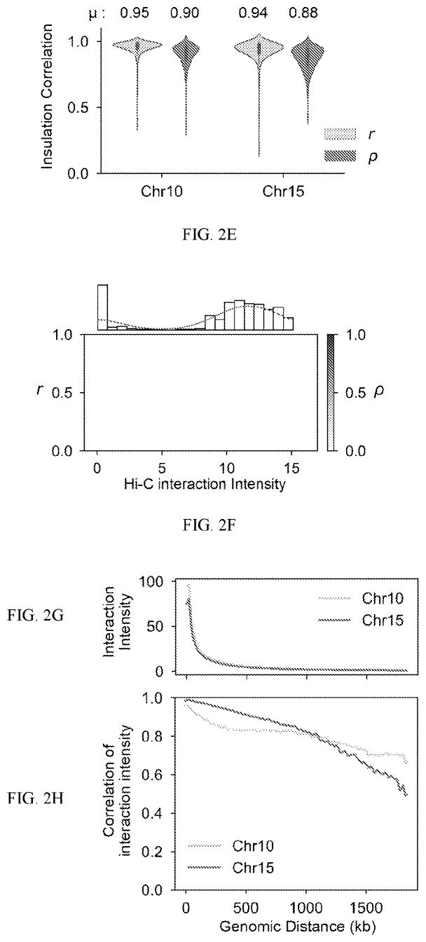

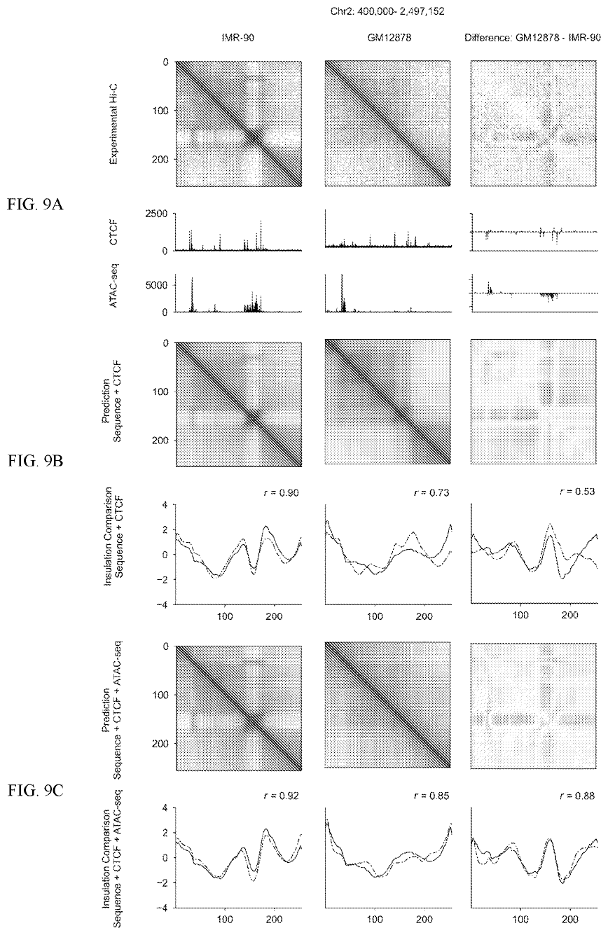

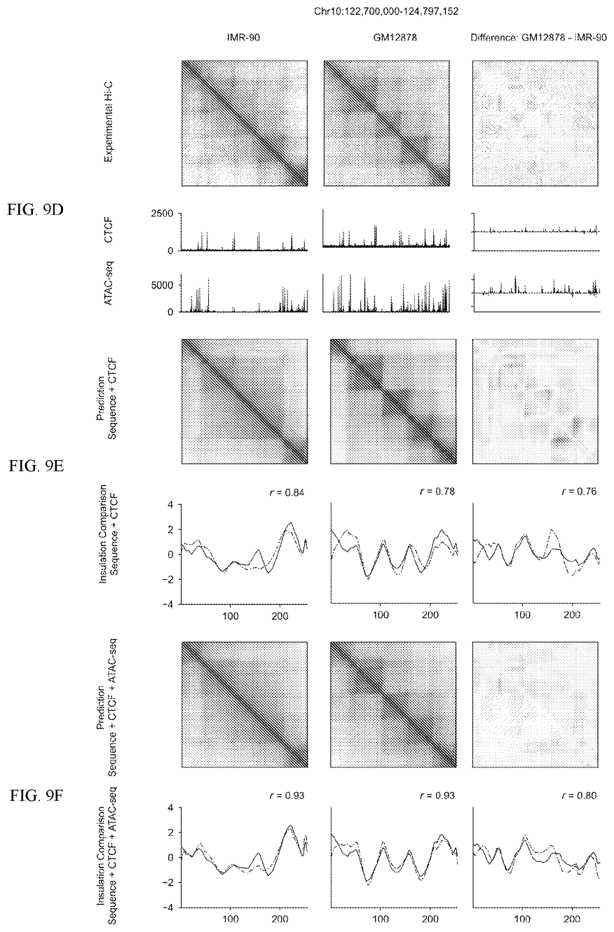

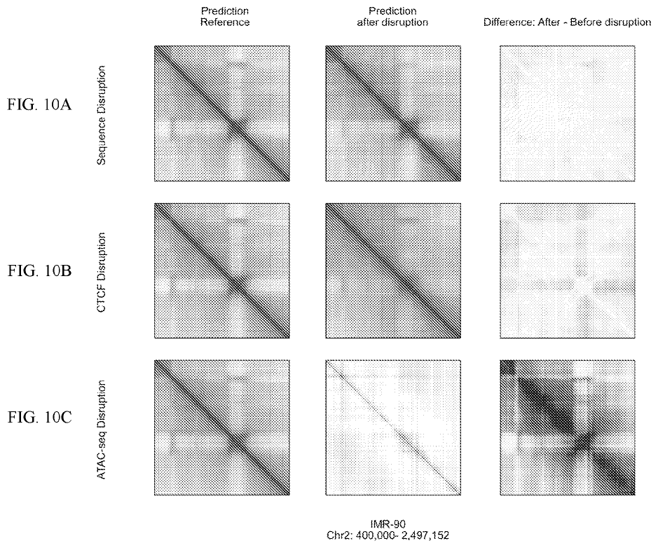

Previous results indicate that chromatin accessibility directly or indirectly affects genome conformation with cell type-specific interactions. We thus improved the model by including ATAC-seq signals to represent chromatin features (FIG. 1B). We found that C.Origami trained with nucleotide-level DNA sequence, CTCF ChIP-seq, and ATAC-seq signals provided high-quality predictions for genome conformation (FIGS. 2A-2H). On validation chromosome 10 and test chromosome 15, C.Origami predicted highly accurate contact matrices that emphasized both large topological domains and detailed chromatin looping events (FIGS. 2A-2C and FIGS. 9A-9F). To quantify prediction performance, we calculated the insulation scores from the predicted Hi-C matrix and found a high correlation with the insulation scores calculated from the experimental data (FIG. 2D). C.Origami achieved on average 0.95 and 0.94 Pearson correlation coefficients on validation and test chromosomes, respectively (FIG. 2E). DNA sequence, CTCF binding signal, and chromatin accessibility signal were all required to accurately predict Hi-C contact matrix with high-quality. Shuffling or ablating any of the signals led to compromised prediction (FIGS. 10A-10C).

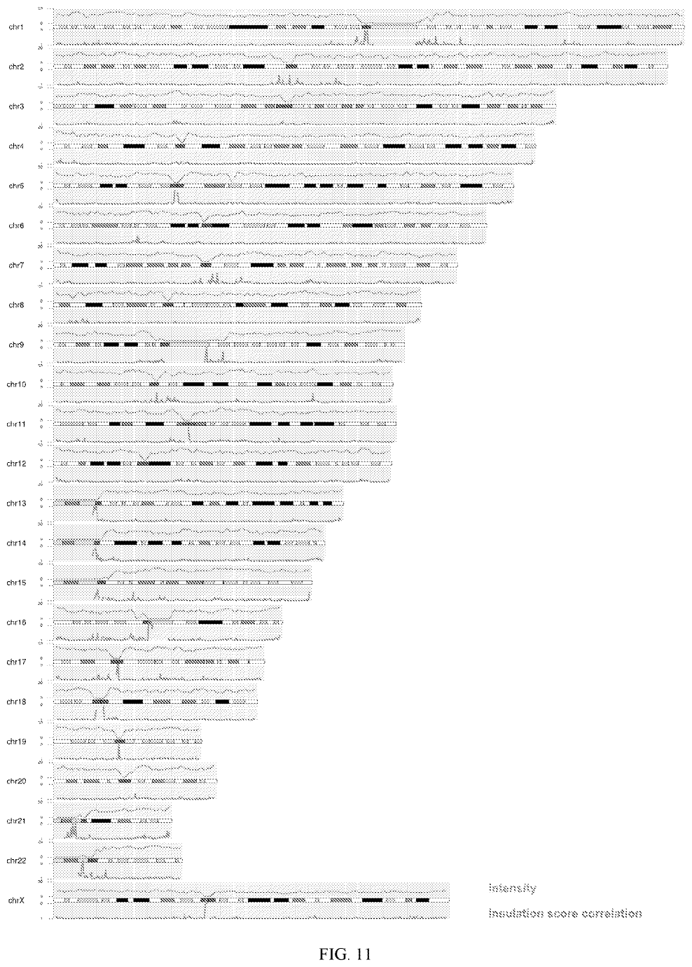

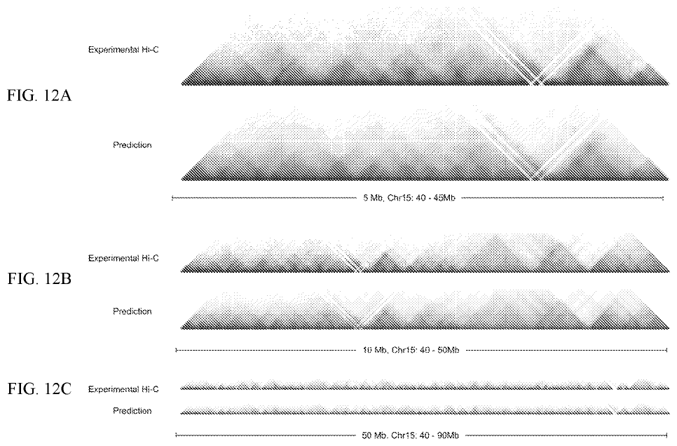

We carried out multiple different measurements to test the performance of our predictive model. First, by plotting the insulation correlation between prediction and experiment against Hi-C data intensity, we found that the predictions in the test set maintain uniform high performance across different clusters, demonstrating robust prediction (FIG. 2F). The few data points with low intensity are regions corresponding to unmappable or repeat sequences such as centromeres and telomeres (FIG. 2F and FIG. 11). Second, our predicted Hi-C contact map followed the exponential decay pattern that generally presents in experimental Hi-C data (FIG. 2G). Third, we plotted the distance-stratified interaction correlation (Pearson) between prediction and experiment. C.Origami achieved correlation above 0.8 within 1000 kb region and 0.6 at 1500 kb (FIG. 2H). Last, we found that predictions from C.Origami were highly consistent across neighboring regions (FIGS. 12A-12C). Thus, C.Origami can be used to construct chromosome-wide prediction of Hi-C contact matrix by joining predictions across sliding windows.

### De Novo Prediction of Cell Type-Specific Chromatin Architecture.

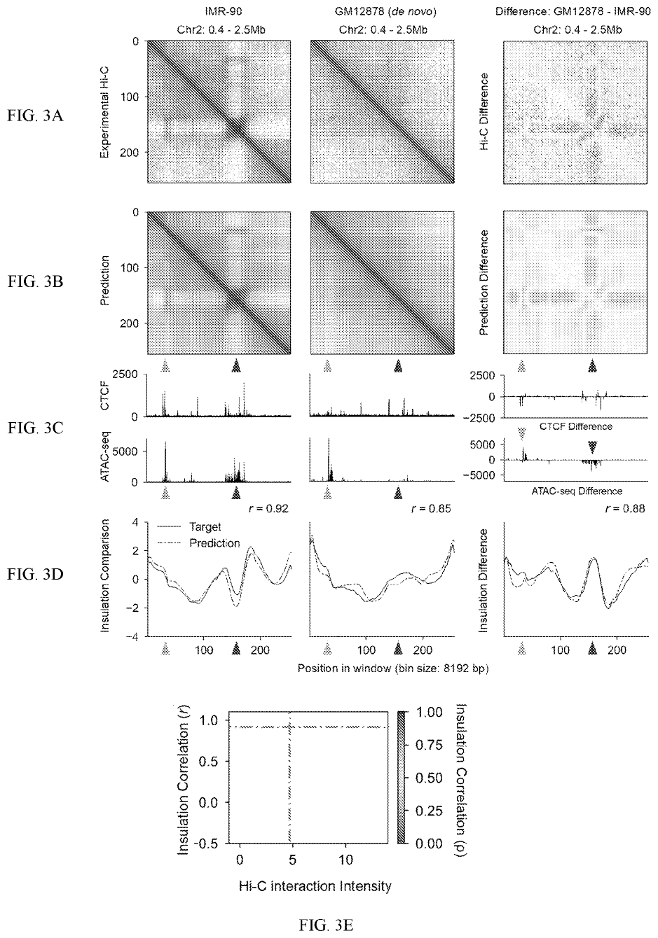

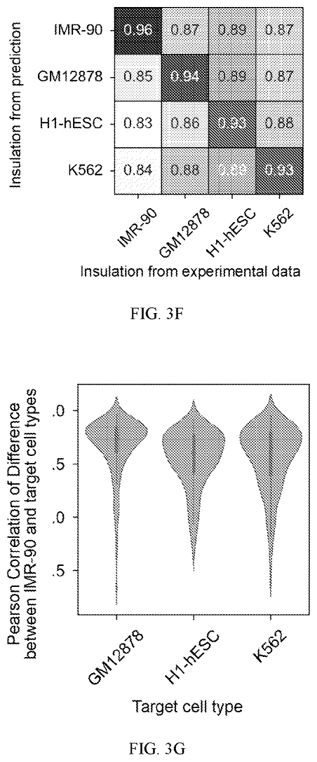

We next tested whether our model generalizes to de novo prediction of chromatin architecture in new cell types. GM12878, a lymphoblastoid cell line, differs substantially from IMR-90 in its chromatin architecture, as exemplified at locus Chr2:400,000-2,497,152 (FIG. 3A). Specifically, we highlight a cell type-specific interaction related to chromatin accessibility changes (black arrowhead) and a distal interaction that associates with both CTCF and ATAC-seq signal changes (gray arrowhead, FIG. 3C). These changes can be clearly demonstrated by differences in their signal intensity (FIGS. 3A and 3C, right). To demonstrate how C.Origami performs in predicting cell type-specific chromatin architecture, we first applied the prediction to both cell types at this locus. We found the cell type-specific chromatin interactions were clearly captured in our prediction, and matched with the experimental Hi-C contact matrix (FIG. 3B). The calculated insulation scores from the predicted Hi-C matrix were also highly correlated with the scores from the experimental data (FIG. 3D). In addition, the difference between insulation scores of the two cell types were highly correlated (FIG. 3D, right), demonstrating that our model not only makes accurate de novo predictions across cell types, but does so with high specificity.

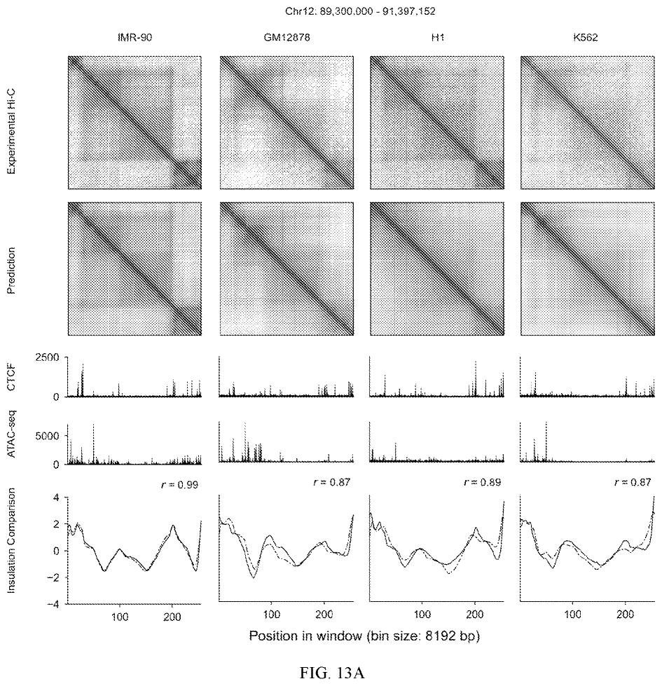

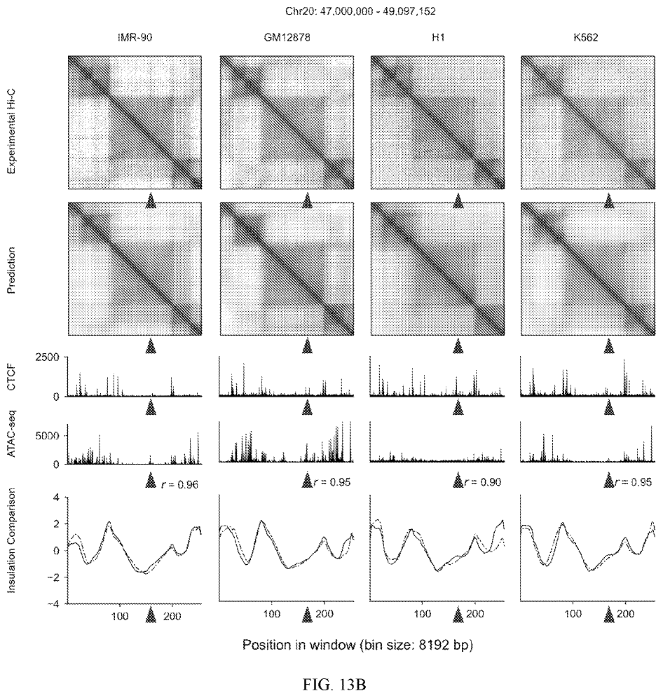

High quality of cell type-specific prediction was achieved across multiple cell types. We further demonstrated the quality of the prediction with two more cell lines, embryonic H1-hESC and erythroleukemia K562. Again, our model accurately predicted the cell type-specific chromatin architecture with high specificity, demonstrating the robustness of C.Origami in de novo prediction and its practical potential for general application (FIGS. 13A-13B).

We next proceeded to systematically assess the performance of our model across the genome. Although we presented multiple loci that have distinct chromatin structures between cell types, most TAD boundaries are conserved across cell types. Thus, to effectively evaluate the capability of C.Origami for cell type-specific prediction, we first performed a filtering analysis to focus on the differentially organized chromatin regions. Regions with normal intensity (>10% intensity quantile) and low similarity (<20% insulation difference) between the experimental Hi-C matrices of the two cell types were selected, totaling ˜15% of the total loci (˜450 Mb of the genome) for testing the performance of predicting cell type-specific chromatin folding (FIG. 3E).

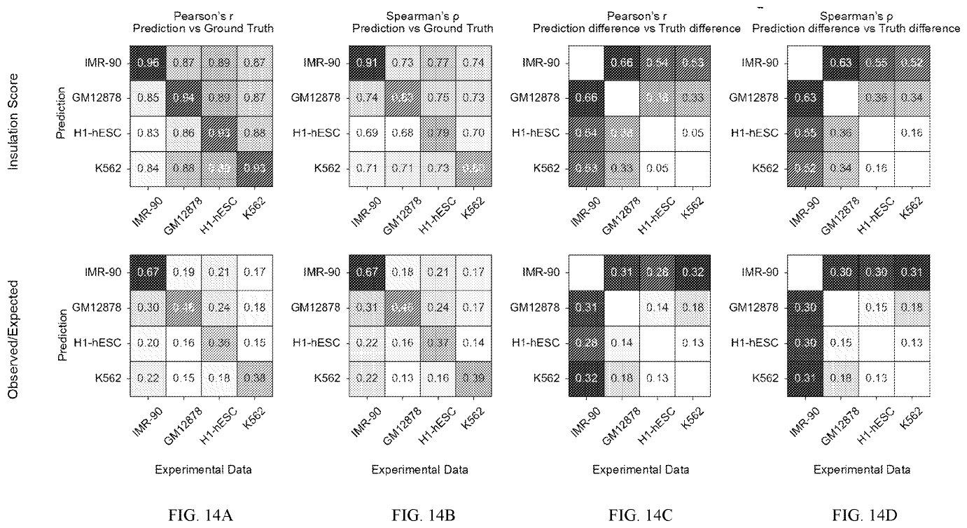

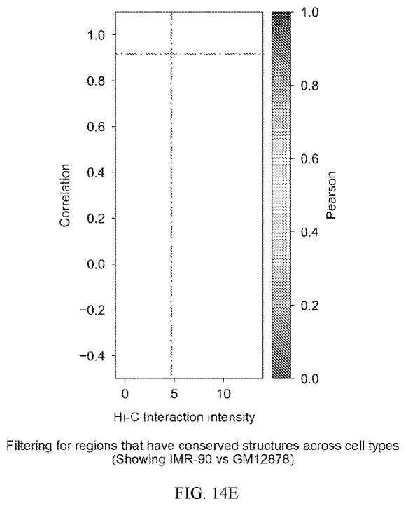

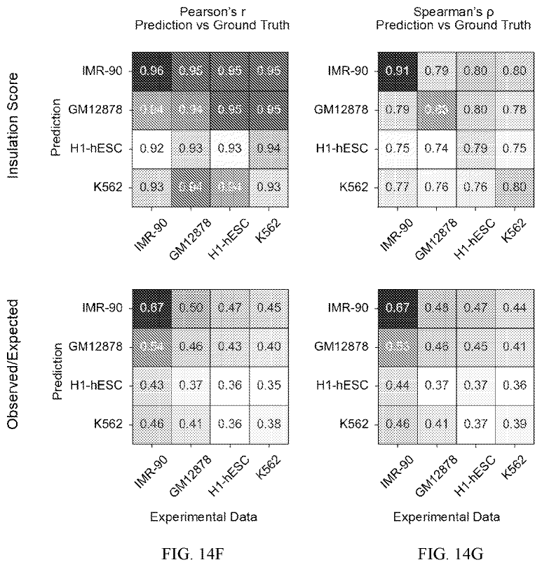

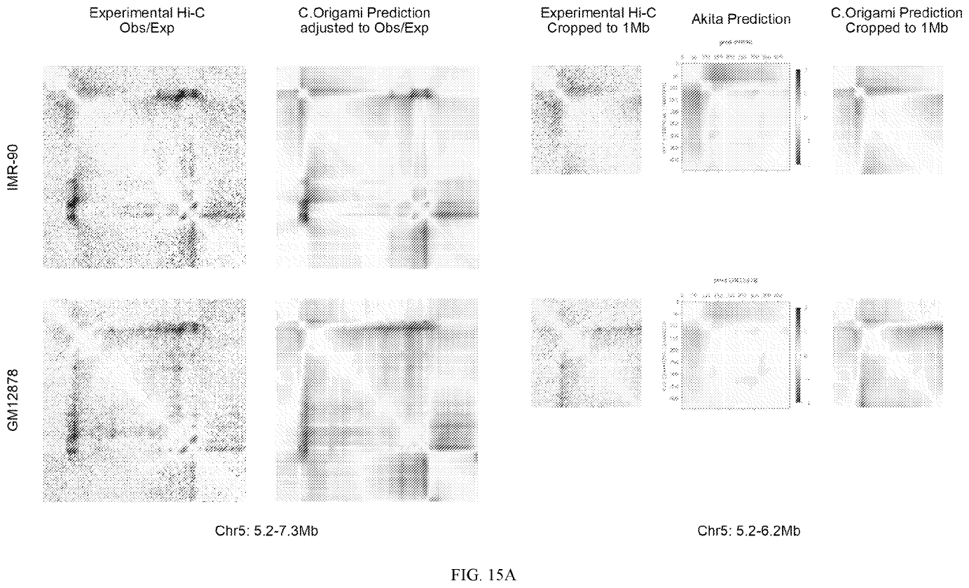

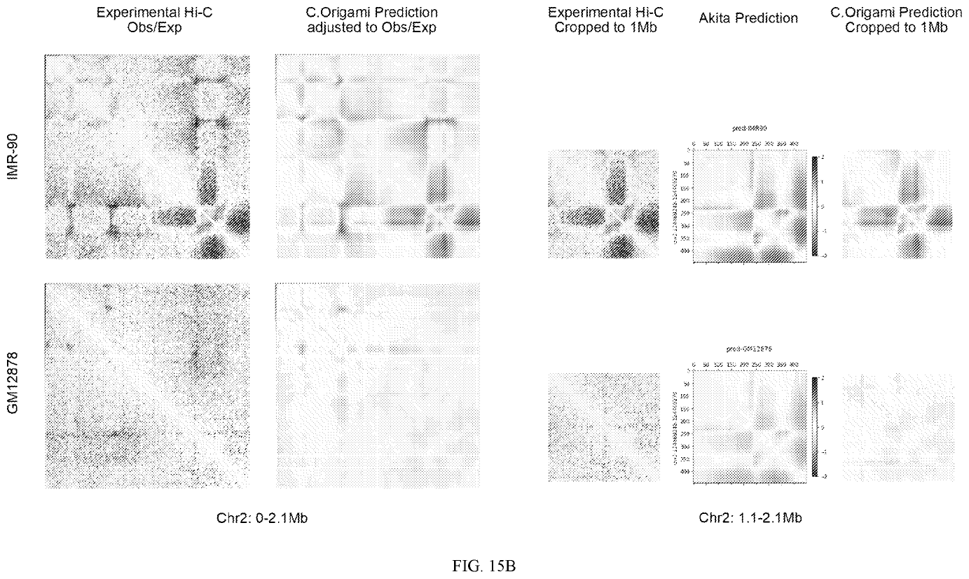

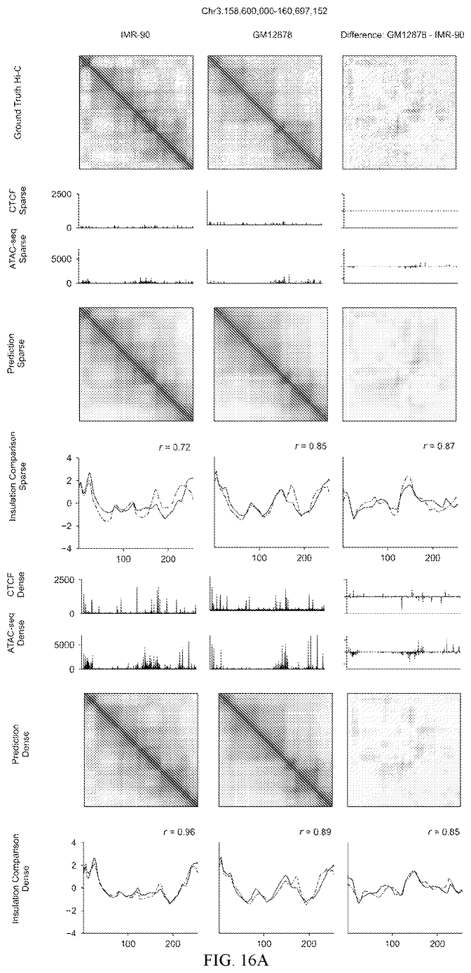

We calculated the correlation coefficient between the insulation scores of the predicted and ground truth Hi-C matrices across all four cell types. In line with observations from the single locus experiment (FIGS. 3A-3D), we found that predictions of one cell type correlated significantly higher with its corresponding experimental data (scores at the diagonal line, FIG. 3F). The correlation coefficients between prediction and experimental data from different cell types were lower, consistent with the expectation that the model predicts cell type-specific chromatin interactions (FIG. 3F). Similarly, these results were recapitulated by correlation analysis using pixel-level Observed/Expected contact matrices (FIGS. 14A-14B).

As a control, we performed a similar analysis using structurally conserved genomic regions, characterized by normal intensity (>10% intensity quantile) and high similarity (>20% insulation difference), across the two cell types (FIG. 14E). As expected, we found the prediction in these regions was highly correlated with the experimental data across cell types (FIGS. 14F-14G).

To quantify the performance of C.Origami in predicting cell type-specific chromatin architecture across the genome, we calculated the insulation difference between predicted Hi-C matrices a pair of cell types. We then computed the correlation between the insulation difference of predictions and that of experimental data. For simplicity, we used IMR-90 as the reference to compare against all the other cell lines. Again, we found all comparisons yielded high correlations between prediction and experimental data (FIG. 3G, FIGS. 14C-14D).

We further compared the performance of C.Origami to Akita, a deep learning model trained on DNA sequence alone for predicting Hi-C contact matrix. We found C.Origami outperformed the Akita model and made accurate cell type-specific predictions regardless of locations (FIGS. 15A-15B). Together, our results indicate that C.Origami trained with DNA sequence along with CTCF binding and chromatin accessibility signals is highly capable of producing de novo predictions of the Hi-C contact matrix, and captures cell type-specific chromatin folding features with high sensitivity.

### Allele-Specific Prediction in Rearranged Cancer Genomes.

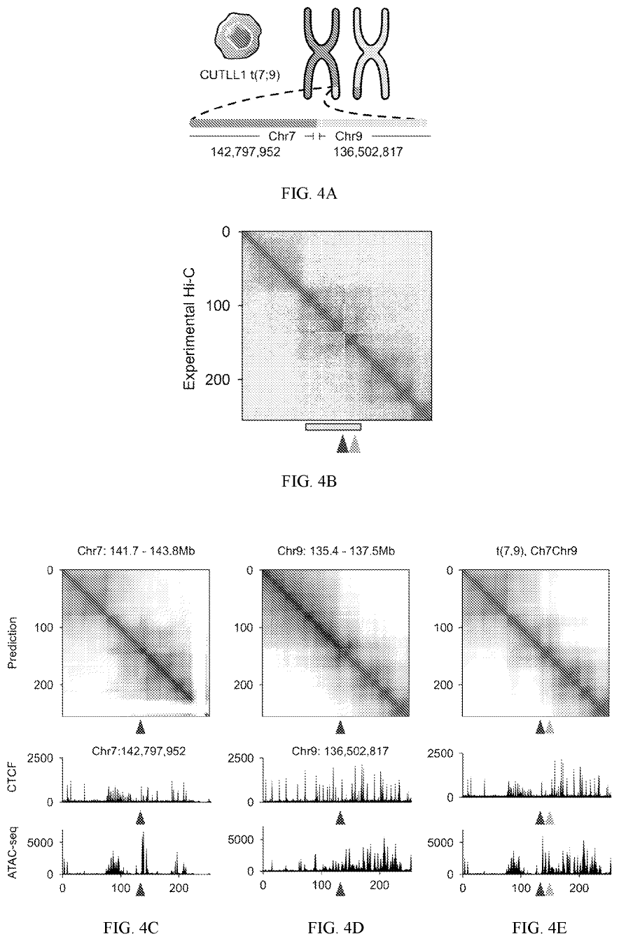

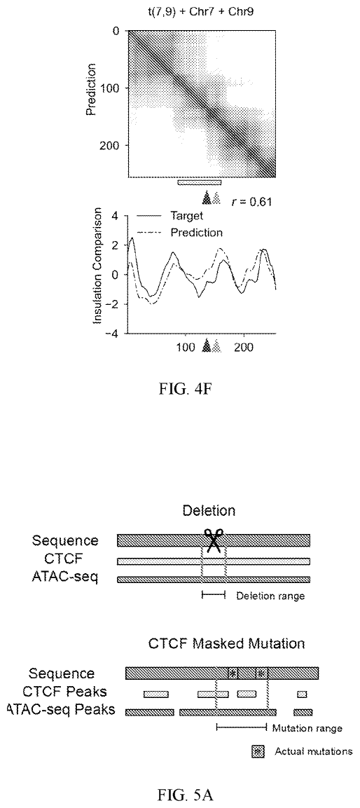

Chromosomal translocations and other structural variants generate novel recombined DNA sequences, subsequently inducing new chromatin interactions which may be critical in tumorigenesis and progression. However, the allelic effect of translocation and structural variations frequently seen in cancer genomes makes it challenging to distinguish the chromatin architecture of the variant chromosome from the normal one. For example, CUTLL1, a T cell leukemia cell line, incorporated a heterozygous t(7,9) translocation where the end of chromosome 7 is recombined with chromosome 9 (FIG. 4A). The translocation introduces new CTCF binding signals from chromosome 9 to chromosome 7. Experimental Hi-C in CUTLL1 cells detected the formation of a neo-TAD at the translocation locus when mapped to a custom CUTLL1 reference genome (FIG. 4B). However, due to the limitation in reference genome mapping, experimental Hi-C usually measures allele-agnostic chromatin architecture, and is thus unable to quantify allele-specific translocation.

To examine the performance of C.Origami in predicting chromatin architecture from recombined cancer genomes, we applied the model to 2 Mb windows centered at the translocation breakpoint in CUTLL1 cells (FIG. 4C-4E). We first predicted the Hi-C contact matrices referring to normal alleles at chromosome 7 and chromosome 9 (FIG. 4C-4D). Since the input CTCF ChIP-seq and ATAC-seq profiles can only be mapped allele-agnostically, our prediction used these inputs as an approximation. Then we simulated the translocation by fusing DNA sequences at the breakpoint in Chromosome 7 (q34) to the Chromosome 9 (q34) breakpoint together with all genomic features (see Methods). The predicted Hi-C map from translocation detected a neo-TAD forming between the two recombined chromosomes (FIG. 4E). Specifically, we found a stripe extending from translocated chromosome 9 to chromosome 7, indicating a novel regulation in the recombined chromosome (FIG. 4E, gray arrowhead). We next averaged the Hi-C contact matrix from normal and translocated alleles, mimicking the allele-agnostic Hi-C mapping in the experimental data, and find a high correlation between the two (FIGS. 4B and 4F, see Methods). The high accuracy in prediction underscores the potential of applying C.Origami in future cancer genomics studies.

Transferring Knowledge Learned from Human Genome to Predict Mouse Chromatin Architecture.

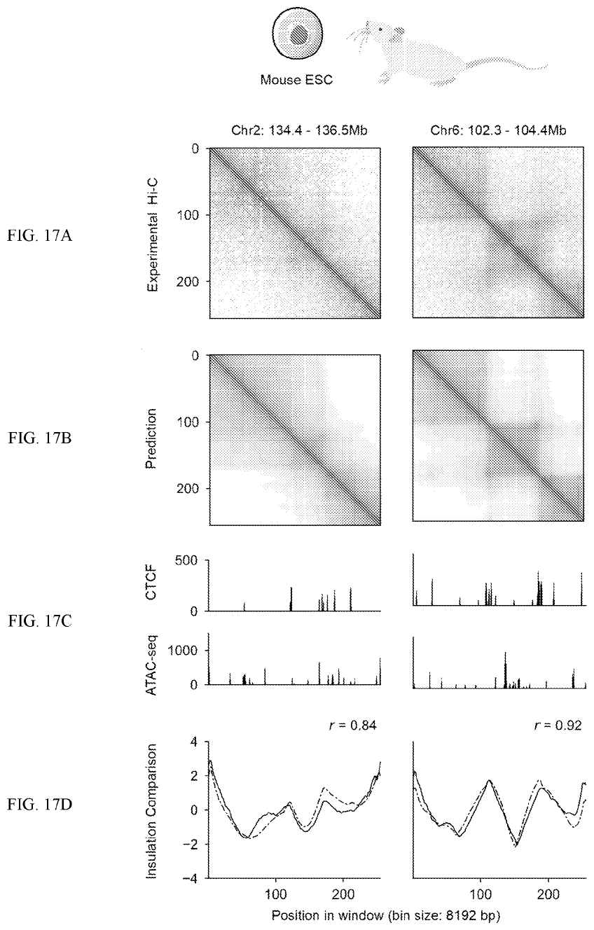

The mouse genome differs from human in its genomic components but the two share a similar mechanism in 3D chromatin organization. We sought to test whether C.Origami could apply knowledge learned from human genome to a different species (FIGS. 17A-17D). In an initial trial, we found that our model trained with DNA sequences and dense genomic features (e.g., bigwig tracks) did not achieve good performance. We hypothesized that the background intensity in dense features can be highly specific to species and the knowledge learned from dense profiles in human could not be transferred to the mouse.

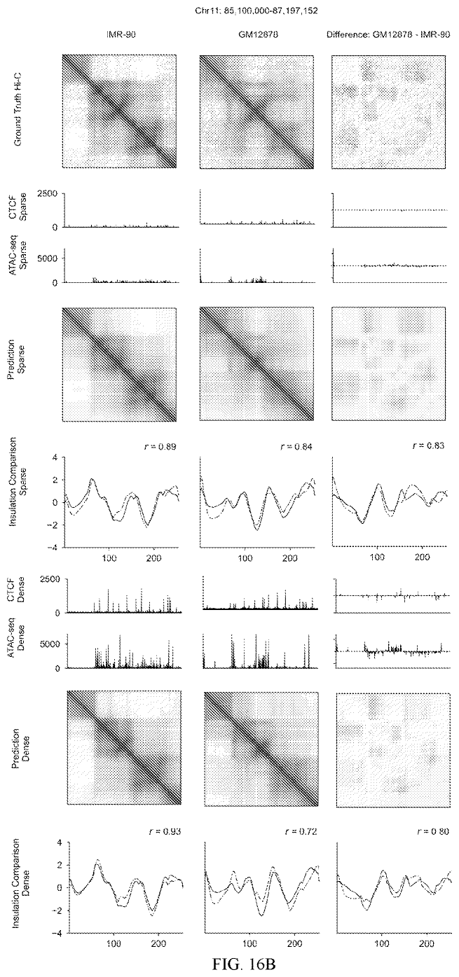

We expect sparse features such as peaks to be less specific, and more consistent across species. To achieve cross-species prediction using a model trained with human data, we slightly modified our input data by performing a peak-calling step on the input genomic profiles and used the sparse genomic features as input for training and prediction (see Methods). The sparse input genomic features did not significantly undermine the performance of the model when repeating the prediction on human cell types comparing to dense features (FIGS. 16A-16B). Applying the model trained on sparse genomic features of human IMR-90 cell line, we found that the model was able to predict mouse chromatin architecture with good quality, indicating the power of C.Origami for transferring the conserved genomic features learned from different species (FIGS. 17A-17D). Notwithstanding the good performance, the accuracy of C.Origami can be further improved by training on mouse data to adapt to mouse chromatin architecture predictions.

High-Accuracy Prediction of C.Origami Enables Cell Type-Specific in silico Genetic Experiments.

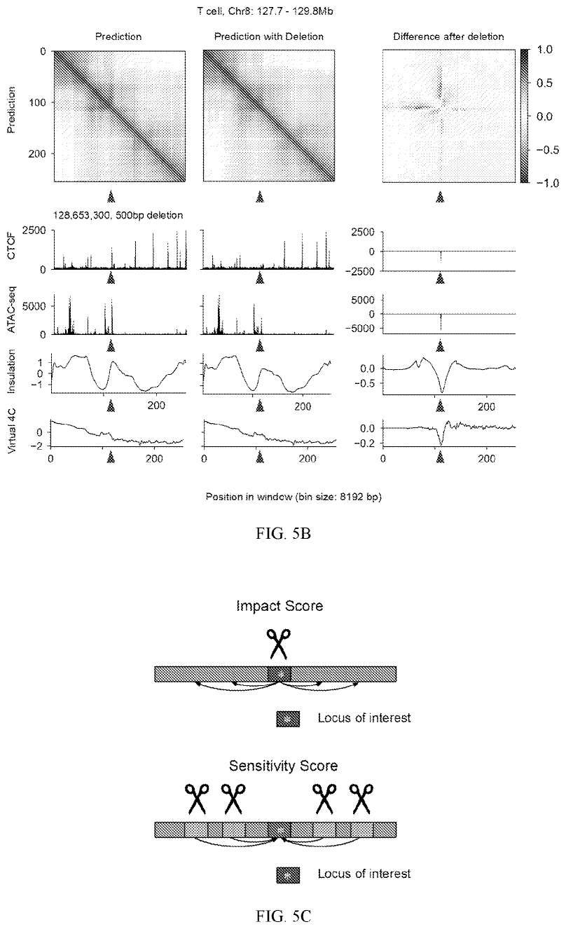

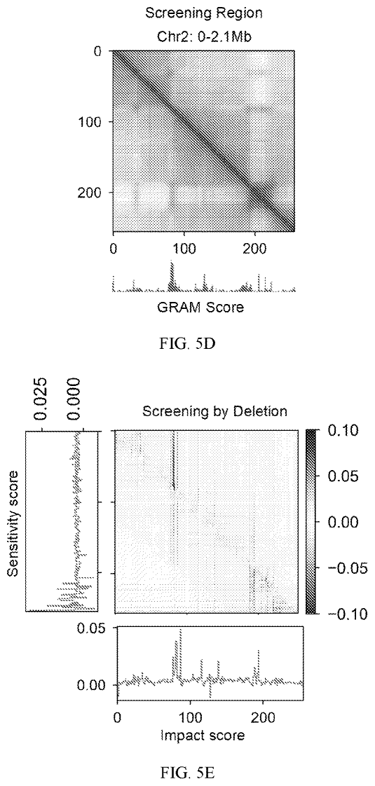

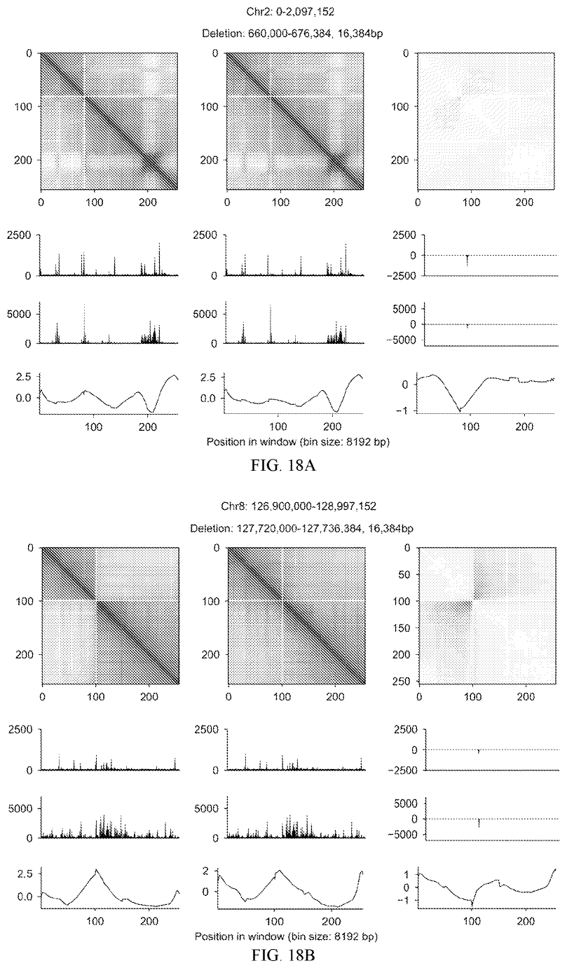

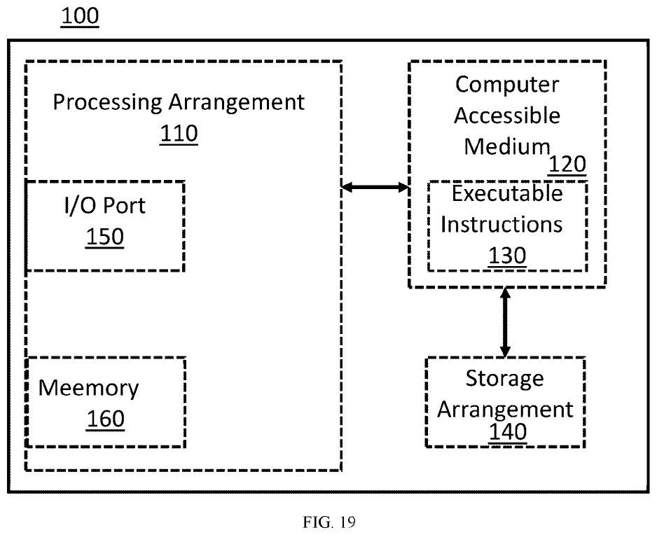

The high accuracy of C.Origami allowed us to perform cell type-specific in silico experiments, and therefore enabled studying how chromatin interaction may be altered upon genetic perturbation. Deletions and mutations are two common types of perturbations in genetic studies. Deletion removes all three types of input features at the perturbed locus, and can thus lead to a TAD merge event (FIG. 5A, top). Instead of experimentally performing such genetic studies, we modelled deletions of TAD boundary sequences in IMR-90 cells in silico, and subsequently predicted local chromatin interaction maps with C.Origami. We found that in silico deletion at TAD boundaries led to TAD merging events of the originally insulated adjacent TADs and a sharp drop in insulation score (FIGS. 18A-18B), indicating the impact of this genetic alteration.

To further investigate the validity of in silico genetic experiments, we applied C.Origami to predict chromatin interactions surrounding the MYC locus which was experimentally perturbed in T cells. Our previous study showed that disrupting a CTCF-binding site near MYC reduced the chromatin looping efficiency in T cells, resulting in a reduced insulation score. Applying C.Origami at the locus, we found a stripe in the predicted Hi-C matrix (FIG. 5b, left, arrowhead), while a 500bp in silico deletion covering the perturbed CTCF-binding signal attenuated such interaction (FIG. 5B, middle and right). Based on our predicted Hi-C matrices, we calculated virtual 4C profiles after perturbing the CTCF binding site and found these to be consistent with the experimental data.

### Cell Type-Specific In Silico Genetic Screen of Cis-Regulatory Elements

To determine whether C.Origami could be used to identify cis-regulatory elements affecting chromatin folding using in silico genetic screening, we first defined a GRAM (Gradient-weighted Regional Activation Mapping) score to estimate how each genomic site contributed to the prediction (FIG. 5C, see Methods). We found GRAM score precisely captured important genomic regions that determine 3D genome structure such as TAD boundaries (FIG. 5C).

To orthogonally demonstrate the capability of C.Origami in discovering novel regulation of chromatin architecture, we carried out in silico genetic screening experiments by systematic perturbation. We divided the window into 256 perturbation regions of ˜8 kb, followed by deletion and prediction across the whole 2 Mb window (see Methods). This process produced a mapping of intensity shift at each perturbed region. We defined the impact score to measure the contribution of a locus on chromatin folding in the 2 Mb window (FIG. 5C, top). This was calculated as the average intensity change of the entire 2 Mb window after perturbation of a given locus. We also defined a sensitivity score to measure how sensitive a locus is to the perturbations of its surrounding regions (FIG. 5C, bottom). We calculated this as the average intensity change of one locus when every region in a 2 Mb window is perturbed.

We found that deletion at TAD boundaries with enriched CTCF ChIP-seq peaks had the highest impact on chromatin folding in the in silico screen experiment (FIG. 5E). This result is consistent with the fact that CTCF binding is a key feature in determining TAD boundaries, and its deletion can lead to alteration of TAD structure, thereby changing the overall intensity of neighboring regions. In contrast, we found sensitivity scores were more similar for loci within the same TADs than those across different TADs, consistent with the expectation that the deletion perturbation is likely to cause intensity shifts within the TAD (FIG. 5E). Similar observations were found across other randomly selected genomic loci (FIG. 11).

To discover CTCF-independent factors regulating chromatin interaction, we performed an in silico screening experiment through CTCF-masked mutagenesis (referred as mutation in later text). CTCF peaks and the corresponding DNA sequences were first masked to keep them intact. We then performed the mutation experiment in a given region by shuffling DNA sequences, followed by a prediction from C.Origami on the 2 Mb genomic window (see Methods). In mutation screening, for the process to yield high impact signal on the locus of interest other than CTCF peaks, DNA sequences and/or ATAC-seq signals must make significant contribution to the final prediction. Mutation screening allowed us to identify multiple CTCF-independent genomic regions that might be critical for chromatin architecture (FIG. 5F). Together, our data show that C.Origami can be used to systematically identify how cis-regulatory elements affect chromatin folding in high-throughput in silico genetic screening.

### Genome-Wide In Silico Screening Revealed Canonical and Novel Regulators of Chromatin Folding.

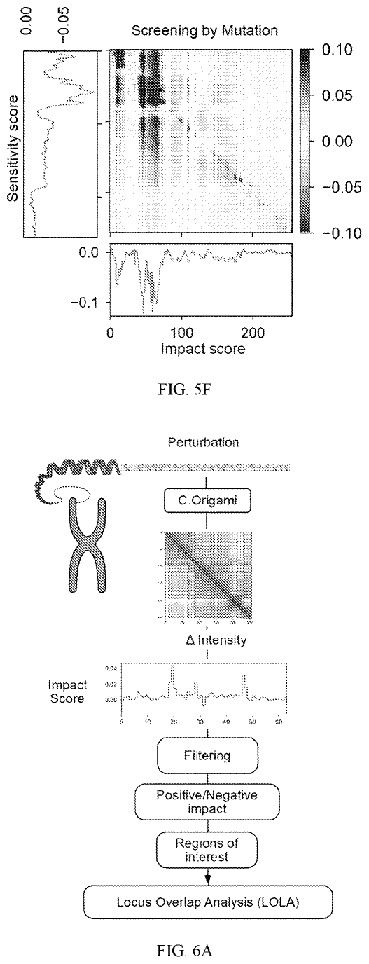

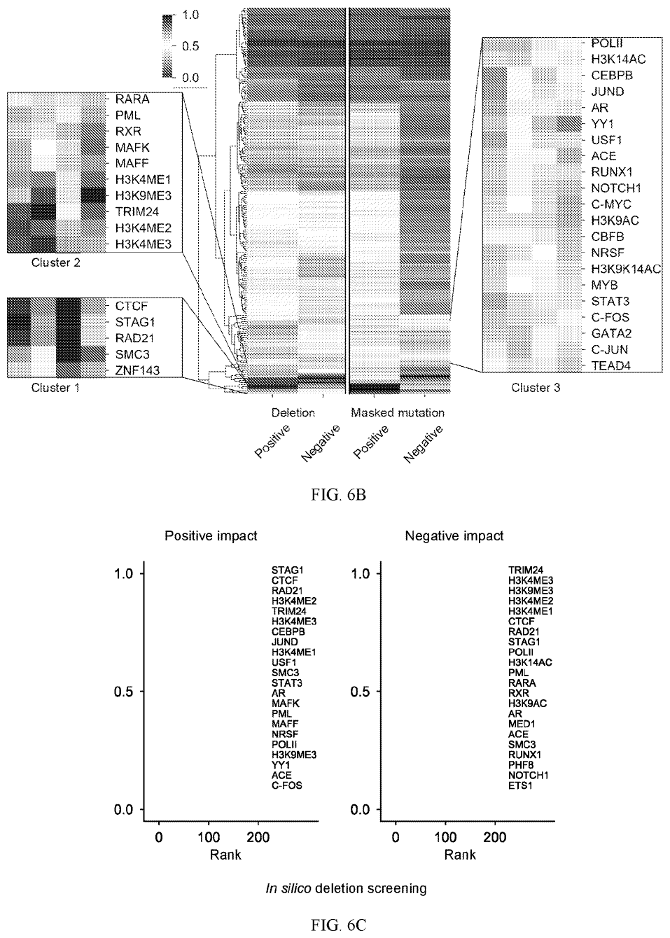

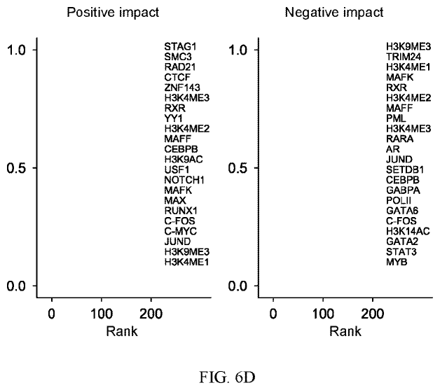

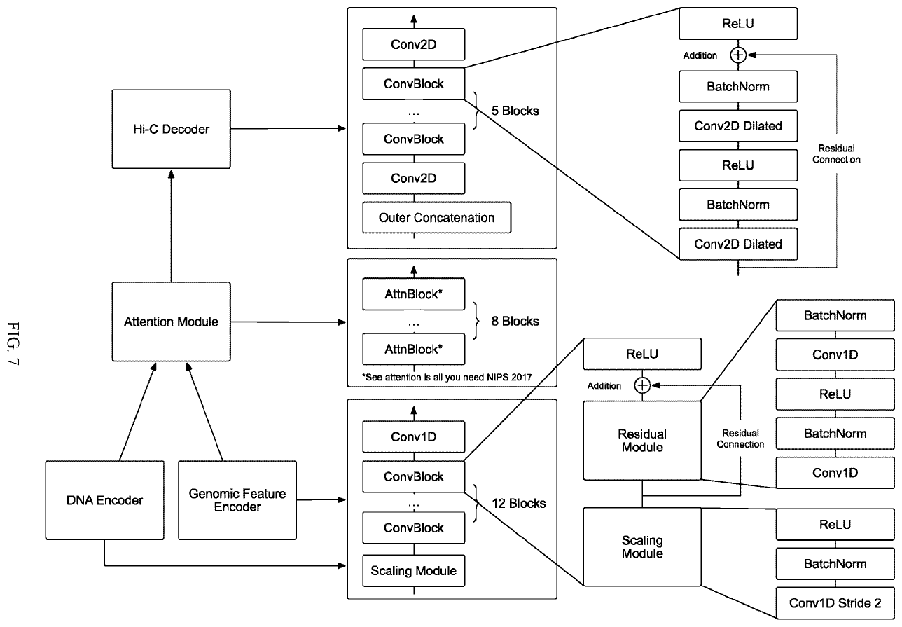

We next asked whether C.Origami could identify a compendium of trans-acting regulators of chromatin interactions in a cell-type specific scenario. We first systematically scanned through the whole genome to discover genomic loci that were critical for predicting chromatin folding in IMR-90 cells. We separately applied in silico deletion and mutation experiments across the entire genome and calculated the impact score at each 20 Kb locus. The DNA sequence of the perturbed loci with high impacts—positive or negative—were designated as potential functional elements for subsequent analysis with LOLA (Locus OverLap Analysis for enrichment of genomic ranges) (FIG. 6A).

Scanning throughout the genome separately in the two types of in silico screen allowed us to identify factors important for chromatin folding (FIG. 6B). As expected, CTCF, together with other canonical factors such as RAD21, STAG1 and SMC3, were significantly enriched in the positive impact score categories due to their role in generating TAD domains (FIG. 6B, cluster 1). These factors did not stand out in the negative score category of mutation screening due to CTCF masking, acting as a negative control for the results.

In contrast to the category enriched in the positive impact score group, we identified a cluster of factors which strongly associated with both positive and negative impacts on chromatin folding in the screening experiments (FIG. 6B, cluster 2). Of note, this cluster was enriched in several histone modifications represented by H3K4me1/2/3, identifying active chromatin marks that are known to contribute to enhancer-promoter looping. This cluster is also enriched for H3K9me3, a mark of constitutive heterochromatin, which is involved in shaping chromatin compartment boundaries.

In addition, the in silico screening identified multiple transcription factors which may function to modulate fine-scale chromatin interactions. The positive impact score categories enriched for many transcription factors (FIG. 6B, cluster 3), such as YY1, NOTCH, and GATA2, indicating that the in silico screening precisely identified these as critical factors for chromatin interactions, in line with previous studies. Beyond this, cluster 3 identified factors that were not previously known to have a role in in modulating chromatin interactions, such as the stress response transcription factors JUND and C-JUN. Interestingly, other AP-1 family proteins such as FOS, have been reported to alter chromatin interactions of their targeting genes. Together, our in silico genetic screen confidently recognized critical chromatin architecture regulators, highlighting its potential for identifying a compendium of trans-acting factors and discovering novel regulation in determining chromatin interactions.

### Discussion

Cell type-specific gene expression profiles require unique chromatin folding patterns. In this study, we developed a novel deep neural network model, C.Origami, that synergistically incorporates both DNA sequence and cell type-specific genomic features for de novo prediction of genome structure.

We found that CTCF binding together with DNA sequence was not sufficient for accurately predicting cell type-specific chromatin architecture. Additional features such as cell type-specific chromatin states play an essential role in chromatin interactions. Consistent with this, we found that incorporation of ATAC-seq data into C.Origami provided enough information for accurate prediction of cell type-specific chromatin interactions, mirroring the results of a high-quality Hi-C experiment. It is worth mentioning that, while preparing the manuscript, another method, Epiphany, was developed for cell type-specific prediction of Hi-C contact matrices using five input genomic profiles. Compared with Epiphany, C.Origami achieved high-quality prediction while using less input data, which greatly expands its practical applications.

With highly accurate prediction of chromatin architecture, our model enables in silico genetic perturbation as a tool to discover functional cis-elements and trans-acting regulators which together determine 3D chromatin architecture in a cell type-specific manner. We performed genome-wide in silico screening of features using deletion and masked mutation experiments in IMR-90 cells. This in silico screening not only identified canonical factors for determining chromatin architecture, such as CTCF, RAD21, STAG1 and SMC3, but also novel transcription factors that potentially function through modulating fine-scale chromatin structure for regulation of gene expression. Meanwhile, the flexible scoring criteria enables unbiased screening for more targeted feature discovery, thus further empowering future genomics studies. We postulate that systematic in silico screening could be generally applicable in discovering novel 3D genome regulatory mechanisms and identifying the specific compendium of regulators across different cell types.

We demonstrated that by integrating cell type-specific genomic features and DNA sequence features, C.Origami model is capable of predicting complex genomic features such as 3D chromatin architecture with high accuracy. The underlying architecture of our model, Origami, is generalizable beyond 3D genome structure prediction. Ultimately, Origami can be trained with appropriate genomic datasets for predicting cell type-specific genomic features, such as epigenetic modifications. We expect future genomics study to shift towards using tools that leverage high-capacity machine learning models to perform in silico experiments for discovering novel genomic regulation.

### Definitions

No claim element herein is to be construed under the provisions of 35 U.S.C. § 112(f), unless the element is expressly recited using the phrase “means for.”

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any references herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

As shown in FIG. 15, e.g., a computer-accessible medium 120 (e.g., as described herein, a storage device such as a hard disk, floppy disk, memory stick, CD-ROM, RAM, ROM, etc., or a collection thereof) can be provided (e.g., in communication with the processing arrangement 110). The computer-accessible medium 120 may be a non-transitory computer-accessible medium. The computer-accessible medium 120 can contain executable instructions 130 thereon. In addition, or alternatively, a storage arrangement 140 can be provided separately from the computer-accessible medium 120, which can provide the instructions to the processing arrangement 110 so as to configure the processing arrangement to execute certain exemplary procedures, processes and methods, as described herein, for example. The instructions may include a plurality of sets of instructions. For example, in some implementations, the instructions may include instructions for applying radio frequency energy in a plurality of sequence blocks to a volume, where each of the sequence blocks includes at least a first stage. The instructions may further include instructions for repeating the first stage successively until magnetization at a beginning of each of the sequence blocks is stable, instructions for concatenating a plurality of imaging segments, which correspond to the plurality of sequence blocks, into a single continuous imaging segment, and instructions for encoding at least one relaxation parameter into the single continuous imaging segment.

System 100 may also include a display or output device, an input device such as a key-board, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Although the figures and description may illustrate a specific order of method steps, the order of such steps may differ from what is depicted and described, unless specified differently above. Also, two or more steps may be performed concurrently or with partial concurrence, unless specified differently above.

1. A method of predicting 3D genomic features in a target cell, the method comprising:
training a neural network model architecture integrating
(1) nucleotide-level DNA sequences, and
(2) cell type-specific genomic features, wherein the cell type-specific genomic features comprise
(i) genomic DNA-binding protein binding profile information, and
(ii) chromatin accessibility information, thereby generating a trained neural network model architecture;
applying the trained neural network model architecture to a genomic window of a target cell; and
identifying genomic features within the genomic window of the target cell.

2. The method of claim 1, wherein the nucleotide-level DNA sequences comprise a naturally occurring wild type sequence, a mutated DNA sequence, or a synthetic DNA sequence.

3. The method of claim 1, wherein the cell type-specific genomic features comprise DNA binding profile information obtained for (1) transcription factor proteins, chromatin binding proteins, and chromatin-associated proteins, or from (2) chromatin feature distribution profiles.

4. The method of claim 3, wherein the chromatin feature distribution profiles comprise histone modifications, DNA modifications, chromatin accessibility information.

5. The method of claim 1, wherein the genomic DNA-binding protein is selected from the group consisting of CTCF, CTCFL, RAD21,STAG1, STAG2, SMC1, SMC3, ZNF143, YY1, NIPBL, WAPL, TRIM22, and BATF.

6. The method of claim 1, wherein the genomic DNA-binding protein is CTCF.

7. The method of claim 1, wherein the genomic DNA-binding protein binding profile information comprises, ChIP-seq data, CUT&RUN data, CUT&TAG data, or DamID data in the genomic window of the target cell.

8. The method of claim 1, wherein the cell type-specific genomic features comprise chromatin feature distribution profiles.

9. The method of claim 1, wherein the chromatin feature distribution profiles comprise histone modification data, DNA modification data.

10. The method of claim 1, wherein the chromatin accessibility information comprises one or more of H3K4ac, H3K9ac, H3K27ac, H3K4mel, H3K4me2, H3K4me3, H3K9me3, H3K27me3, H3K36me3.

11. The method of claim 1, wherein the chromatin accessibility information is selected from the group consisting of ATAC-seq data, DNase-seq data, or MNase-seq data.

12. The method of claim 1, wherein the cell type-specific genomic comprises a DNA modification profile.

13. The method of claim 12, wherein the DNA modification profile comprises DNA methylated cytosine (5mC), DNA hydroxylmethylaed cytosine (5hmC), or DNA formylated cytosine (5hmC), or carboxylated cytosine (5caC).

14. The method of claim 11, wherein the chromatin accessibility information comprises ATAC-seq data in the genomic window of the target cell.

15. The method of claim 1, wherein genomic features comprise identification of a topologically associating domain (TAD).

16. The method of claim 1, wherein the genomic window comprises a contiguous genomic region of 2 million bases.

17. The method of claim 1, wherein the model architecture comprises two encoders, a transformer module, and a decoder.

18. The method of claim 17, wherein the decoder is a decoder associated with Hi-C contact matrices for predicting complex chromatin architecture.

19. A computer-implemented machine for predicting 3D genomic features in a target cell, comprising:
a processor;
a neural network comprising a first encoder, a second encoder, a transformer module, and a decoder; and
a tangible computer-readable medium operatively connected to the processor and including computer code configured to:
train a neural network model architecture integrating
(1) nucleotide-level DNA sequences, and
(2) cell type-specific genomic features, wherein the cell type-specific genomic features comprise
(i) genomic DNA-binding protein binding profile information, and
(ii) chromatin accessibility information,
thereby generating a trained neural network model architecture;
apply the trained neural network model architecture to a genomic window of a target cell; and
identify genomic features within the genomic window of the target cell.

20-38. (canceled)

39. A method of predicting 3D genomic features in a target cell, the method comprising:
training a neural network model architecture integrating
(1) nucleotide-level DNA sequences, and
(2) cell type-specific genomic features, wherein the cell type-specific genomic features comprise
(i) genomic DNA-binding protein binding profile information, and
(ii) chromatin feature profile information,
thereby generating a trained neural network model architecture;
applying the trained neural network model architecture to a genomic window of a target cell;
identifying genomic features within the genomic window of the target cell; and
screening for genomic features across the genomes of multiple cell types in silico.
